(19) United States
(12) Patent Application Publication
(10) Pub. No.: US 2017/0327786 A1
(43) Pub. Date: Nov. 16, 2017

(54) Hypotaurine, GABA, Beta-Alanine, and Choline for Control of Waste Byproduct Accumulation in Mammalian Cell Culture Process

(71) Applicant: Biogen MA Inc., Cambridge, MA (US)

(72) Inventors: Yao-Ming HUANG, Cary, NC (US); William C. YANG, Cary, NC (US); Alan GILBERT, Arlington, MA (US); Kyle MCELEARNEY, Medford, MA (US)

(73) Assignee: Biogen MA Inc., Cambridge, MA (US)

(21) Appl. No.: 15/523,009

(22) PCT Filed: Oct. 30, 2015
(86) PCT No.: PCT/US2015/058515

§ 371 Date: Apr. 28, 2017

Related U.S. Application Data
(60) Provisional application No. 62/073,300, filed on Oct. 31, 2014.

(51) Int. Cl. C12N 5/00; C07K 16/28; C07K 14/565; C07K 14/705; C07K 16/24; C07K 16/18; C07K 16/42; C07K 14/525; C12P 21/00; C07K 16/00

(52) U.S. Cl. CPC C12N 5/0018; C12P 21/00; C07K 16/2887; C07K 14/565; C07K 14/70575; C07K 16/00; C07K 16/18; C07K 16/42; C07K 14/525; C07K 16/2839; C07K 16/246; C07K 16/241; C07K 2317/14; C12N 2500/33; C12N 2500/38; C12N 2510/02; C12N 2511/00

(57) ABSTRACT

The present invention pertains to a cell culture medium comprising hypotaurine, GABA, and/or beta-alanine or the combination of choline and hypotaurine, GABA, and/or beta-alanine as media supplements which is shown to control viability, growth, and waste byproduct accumulation. The present invention further pertains to a method of producing a polypeptide of interest in a large scale cell culture containing hypotaurine, GABA, and/or beta-alanine or the combination of choline and hypotaurine, GABA, and/or beta-alanine.

### BACKGROUND OF THE INVENTION

### Field of the Invention

The present invention pertains to a cell culture medium comprising as media supplements hypotaurine, Gamma-Aminobutyric Acid (GABA), and/or β-alanine (beta-alanine), or further in combination with choline, and methods of use thereof. The present invention further pertains to a method of controlling or manipulating production of a polypeptide of interest in a large scale cell culture, comprising controlling or manipulating the concentration of hypotaurine, GABA, and/or beta-alanine, or further in combination with choline in the cell culture medium.

### Background Art

Over the last few decades, much research has focused on the production of therapeutic recombinant proteins, e.g., monoclonal antibodies. While media containing sera or hydrolysates has been utilized, chemically defined media were also developed in order to eliminate the problematic lot-to-lot variation of complex components (Luo and Chen, Biotechnology and Bioengineering 97(6):1654-1659 (2007)). An improved understanding of cell culture has permitted a shift to chemically defined medium without compromising growth, viability, titer, etc. To date optimized chemically defined processes have been reported with titers as high as 7.5-10 g/L (Huang et al., Biotechnology Progress 26(5):1400-1410 (2010); Ma et al., Biotechnology Progress 25(5):1353-1363 (2009); Yu et al., Biotechnology and Bioengineering 108(5):1078-1088 (2011)). In general, the high titer chemically defined processes are fed batch processes with cultivation times of 11-18 days. The process intensification has been achieved without compromising product quality while maintaining relatively high viabilities. However, such cultures have historically been plagued by the accumulation of toxic waste products, such as ammonium and lactate. Thus, there is a need for a method to reduce waste accumulation in cell cultures.

### BRIEF SUMMARY OF THE INVENTION

In one aspect, the present invention is directed to a method of producing a polypeptide of interest in a large-scale cell culture, comprising culturing mammalian cells expressing the polypeptide of interest in a cell culture medium under conditions that support expression of the polypeptide of interest, wherein said cell culture medium comprises hypotaurine, Gamma-Aminobutyric Acid (GABA), and/or beta-alanine or further in combination with choline. In one embodiment, the cell culture medium comprises between about 0.1 mM and about 500 mM hypotaurine. In one embodiment, the cell culture medium comprises between about 0.1 mM and about 500 mM GABA. In one embodiment, the cell culture medium comprises between about 0.1 mM and about 500 mM beta-alanine.

In another aspect, the invention is directed to a method of producing a polypeptide of interest in a large-scale cell culture, comprising supplementing the culture with a feed medium comprising a sufficient amount of hypotaurine to achieve a hypotaurine concentration in the culture between about 0.1 mM and 500 mM, wherein the culture comprises cells expressing the polypeptide and a medium, and the cells are maintained under conditions that allow for expression of the polypeptide.

In another aspect, the invention is directed to a method of producing a polypeptide of interest in a large-scale cell culture, comprising supplementing the culture with a feed medium comprising a sufficient amount of Gamma-Aminobutyric Acid (GABA) to achieve a GABA concentration in the culture between about 0.1 mM and 500 mM, wherein the culture comprises cells expressing the polypeptide and a medium, and the cells are maintained under conditions that allow for expression of the polypeptide.

In another aspect, the invention is directed to a method of producing a polypeptide of interest in a large-scale cell culture, comprising supplementing the culture with a feed medium comprising a sufficient amount of beta-alanine to achieve a beta-alanine concentration in the culture between about 0.1 mM and 500 mM, wherein the culture comprises cells expressing the polypeptide and a medium, and the cells are maintained under conditions that allow for expression of the polypeptide.

In another aspect, the invention is directed to a method of producing a polypeptide of interest in a large-scale cell culture, comprising: a) providing cells capable of expressing the polypeptide and a hypotaurine-containing cell culture medium; b) supplementing the culture with a feed medium comprising a sufficient amount of hypotaurine to achieve a hypotaurine concentration of between about 0.1 mM to 500 mM; and c) culturing the cells of b) to allow for expression of the polypeptide.

In another aspect, the invention is directed to a method of producing a polypeptide of interest in a large-scale cell culture, comprising: a) providing cells capable of expressing the polypeptide and a Gamma-Aminobutyric Acid (GABA)-containing cell culture medium; b) supplementing the culture with a feed medium comprising a sufficient amount of GABA to achieve a GABA concentration of between about 0.1 mM to 500 mM; and c) culturing the cells of b) to allow for expression of the polypeptide.

In another aspect, the invention is directed to a method of producing a polypeptide of interest in a large-scale cell culture, comprising: a) providing cells capable of expressing the polypeptide and a beta-alanine-containing cell culture medium; b) supplementing the culture with a feed medium comprising a sufficient amount of beta-alanine to achieve a beta-alanine concentration of between about 0.1 mM to 500 mM; and c) culturing the cells of b) to allow for expression of the polypeptide.

In one embodiment, the method further comprises supplementing the culture with a feed medium comprising a sufficient amount of hypotaurine to maintain the hypotaurine concentration in the culture to between about 0.1 mM and about 500 mM. In some embodiments, the feed medium comprises hypotaurine in an amount sufficient to achieve a hypotaurine concentration in the culture of between about 0.1 mM and about 500 mM, between about 0.1 mM and about 400 mM, between about 0.1 mM and about 300 mM, between about 0.1 mM and about 200 mM, between about 0.1 mM and about 100 mM, between about 0.1 mM and about 50 mM, between about 0.1 mM and about 25 mM, between about 10 mM and about 500 mM, between about 20 mM and about 500 mM, between about 50 mM and about 500 mM, between about 100 mM and about 500 mM, between about 200 mM and about 500 mM, between about 10 mM and about 100 mM, between about 50 mM and about 200 mM, between about 100 mM and about 400 mM, between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

In one embodiment, the method further comprises supplementing the culture with a feed medium comprising a sufficient amount of Gamma-Aminobutyric Acid (GABA) to maintain the GABA concentration in the culture to between about 0.1 mM and about 500 mM. In some embodiments, the feed medium comprises GABA in an amount sufficient to achieve a GABA concentration in the culture of between about 0.1 mM and about 500 mM, between about 0.1 mM and about 400 mM, between about 0.1 mM and about 300 mM, between about 0.1 mM and about 200 mM, between about 0.1 mM and about 100 mM, between about 0.1 mM and about 50 mM, between about 0.1 mM and about 25 mM, between about 1 mM and about 25 mM, between about 3 mM and about 20 mM, between about 10 mM and about 500 mM, between about 5 mM and about 70 mM, between about 8 mM and about 65 mM, between about 20 mM and about 500 mM, between about 50 mM and about 500 mM, between about 100 mM and about 500 mM, between about 200 mM and about 500 mM, between about 10 mM and about 100 mM, between about 50 mM and about 200 mM, between about 100 mM and about 400 mM, between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

In one embodiment, the method further comprises supplementing the culture with a feed medium comprising a sufficient amount of beta-alanine to maintain the beta-alanine concentration in the culture to between about 0.1 mM and about 500 mM. In some embodiments, the feed medium comprises beta-alanine in an amount sufficient to achieve a beta-alanine concentration in the culture of between about 0.1 mM and about 500 mM, between about 0.1 mM and about 400 mM, between about 0.1 mM and about 300 mM, between about 0.1 mM and about 200 mM, between about 0.1 mM and about 100 mM, between about 0.1 mM and about 50 mM, between about 0.1 mM and about 25 mM, between about 1 mM and about 25 mM, between about 3 mM and about 20 mM, between about 10 mM and about 500 mM, between about 5 mM and about 70 mM, between about 8 mM and about 65 mM, between about 20 mM and about 500 mM, between about 50 mM and about 500 mM, between about 100 mM and about 500 mM, between about 200 mM and about 500 mM, between about 10 mM and about 100 mM, between about 50 mM and about 200 mM, between about 100 mM and about 400 mM, between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

In one embodiment, the medium further comprises choline. In some embodiments, the feed medium comprises choline in an amount sufficient to achieve a choline concentration in the culture of between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.1 mM and about 0.5 mM, about 0.5 mM and about 10 mM, about 1 mM and about 10 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

In one embodiment, the cells are maintained in a cell culture medium containing hypotaurine at a concentration of about 0.1 mM to 500 mM for between about 1 day and about 20 days. In one embodiment, the cells are maintained in a cell culture medium containing hypotaurine at a concentration of about 0.1 mM to 500 mM for between about 1 day and about 20 days, about 1 day and about 15 days, about 1 day and about 10 days, about 1 day and about 9 days, about 1 day and about 8 days, or about 1 day and about 7 days. In one embodiment, the cell culture medium at the hypotaurine concentration is maintained for at least about 1 day, at least about 2 days, at least 3 days, at least about 4 days, at least about 5 days, at least about 6 days, at least about 7 days, at least about 8 days, at least about 9 days, at least about 10 days, at least about 15 days, or at least about 20 days.

In one embodiment, the cells are maintained in a cell culture medium containing Gamma-Aminobutyric Acid (GABA) at a concentration of about 0.1 mM to 500 mM for between about 1 day and about 20 days. In one embodiment, the cells are maintained in a cell culture medium containing GABA at a concentration of about 0.1 mM to 500 mM for between about 1 day and about 20 days, about 1 day and about 15 days, about 1 day and about 10 days, about 1 day and about 9 days, about 1 day and about 8 days, or about 1 day and about 7 days. In one embodiment, the cell culture medium at the GABA concentration is maintained for at least about 1 day, at least about 2 days, at least 3 days, at least about 4 days, at least about 5 days, at least about 6 days, at least about 7 days, at least about 8 days, at least about 9 days, at least about 10 days, at least about 15 days, or at least about 20 days.

In one embodiment, the cells are maintained in a cell culture medium containing beta-alanine at a concentration of about 0.1 mM to 500 mM for between about 1 day and about 20 days. In one embodiment, the cells are maintained in a cell culture medium containing beta-alanine at a concentration of about 0.1 mM to 500 mM for between about 1 day and about 20 days, about 1 day and about 15 days, about 1 day and about 10 days, about 1 day and about 9 days, about 1 day and about 8 days, or about 1 day and about 7 days. In one embodiment, the cell culture medium at the beta-alanine concentration is maintained for at least about 1 day, at least about 2 days, at least 3 days, at least about 4 days, at least about 5 days, at least about 6 days, at least about 7 days, at least about 8 days, at least about 9 days, at least about 10 days, at least about 15 days, or at least about 20 days.

In one embodiment, the cells are maintained in a cell culture medium containing one or more of hypotaurine, Gamma-Aminobutyric Acid (GABA), and beta-alanine at a concentration of about 0.1 mM to 500 mM for between about 1 day and about 20 days. In one embodiment, the cells are maintained in a cell culture medium containing one or more of hypotaurine, Gamma-Aminobutyric Acid (GABA), and beta-alanine at a concentration of about 0.1 mM to 500 mM for between about 1 day and about 20 days, about 1 day and about 15 days, about 1 day and about 10 days, about 1 day and about 9 days, about 1 day and about 8 days, or about 1 day and about 7 days. In one embodiment, the cell culture medium at the beta-alanine concentration is maintained for at least about 1 day, at least about 2 days, at least 3 days, at least about 4 days, at least about 5 days, at least about 6 days, at least about 7 days, at least about 8 days, at least about 9 days, at least about 10 days, at least about 15 days, or at least about 20 days.

In one embodiment, the culture is supplemented with the feed medium between about 1 and about 20 times. In another embodiment, the culture is supplemented with the feed medium about 1 and about 20 times, between about 1 and about 15 times, or between about 1 and about 10 times. In a further embodiment, the culture is supplemented with the feed medium at least once, at least twice, at least three times, at least four times, at least five times, at least six times, at least seven times, at least eight times, at least nine times, at least ten times, at least 11 times, at least 12 times, at least 13 times, at least 14 times, at least 15 times, or at least 20 times.

In one embodiment, the lactate production of the cells is lower than the lactate production of cells maintained in a culture medium that is substantially free from choline and hypotaurine. In one embodiment, the lactate production of the cells is between about 5% and about 95% lower than the lactate production of cells maintained in a culture medium that is substantially free from choline and hypotaurine. In one embodiment, the lactate production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than the lactate production of cells maintained in a culture medium that is substantially free of choline and hypotaurine.

In one embodiment, the lactate production of the cells is lower than the lactate production of cells maintained in a culture medium that is substantially free from choline and Gamma-Aminobutyric Acid (GABA). In one embodiment, the lactate production of the cells is between about 5% and about 95% lower than the lactate production of cells maintained in a culture medium that is substantially free from choline and GABA. In one embodiment, the lactate production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than the lactate production of cells maintained in a culture medium that is substantially free of choline and GABA.

In one embodiment, the lactate production of the cells is lower than the lactate production of cells maintained in a culture medium that is substantially free from choline and beta-alanine. In one embodiment, the lactate production of the cells is between about 5% and about 95% lower than the lactate production of cells maintained in a culture medium that is substantially free from choline and beta-alanine. In one embodiment, the lactate production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than the lactate production of cells maintained in a culture medium that is substantially free of choline and beta-alanine.

In one embodiment, the lactate production of the cells is lower than the lactate production of cells maintained in a culture medium that is substantially free from one or more of hypotaurine, Gamma-Aminobutyric Acid (GABA), and beta-alanine. In one embodiment, the lactate production of the cells is between about 5% and about 95% lower than the lactate production of cells maintained in a culture medium that is substantially free from one or more of hypotaurine, Gamma-Aminobutyric Acid (GABA), and beta-alanine. In one embodiment, the lactate production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than the lactate production of cells maintained in a culture medium that is substantially free of one or more of hypotaurine, Gamma-Aminobutyric Acid (GABA), and beta-alanine.

In one embodiment, the lactate concentration of the culture is between about 0.1 g/L and about 10 g/L. In one embodiment, the lactate concentration of the culture is between about 0.1 g/L and about 5 g/L, between about 0.1 g/L and about 4 g/L, or between about 0.1 g/L and about 3 g/L. In one embodiment, the lactate concentration of the culture is less than about 6 g/L, about 5 g/L, about 4 g/L, about 3 g/L, about 2 g/L, or about 1 g/L.

In one embodiment, the ammonium production of the cells is lower than the ammonium production of cells maintained in a culture medium that is substantially free of choline and hypotaurine. In a further embodiment, the ammonium production of the cells is between about 5% and about 90% lower than the ammonium production of cells maintained in a culture medium that is substantially free from choline and hypotaurine. In a further embodiment, the ammonium production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than ammonium production of cells maintained in a culture medium that is substantially free from choline and hypotaurine.

In one embodiment, the ammonium production of the cells is lower than the ammonium production of cells maintained in a culture medium that is substantially free of choline and Gamma-Aminobutyric Acid (GABA). In a further embodiment, the ammonium production of the cells is between about 5% and about 90% lower than the ammonium production of cells maintained in a culture medium that is substantially free from choline and GABA. In a further embodiment, the ammonium production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than ammonium production of cells maintained in a culture medium that is substantially free from choline and GABA.

In one embodiment, the ammonium production of the cells is lower than the ammonium production of cells maintained in a culture medium that is substantially free of choline and beta-alanine. In a further embodiment, the ammonium production of the cells is between about 5% and about 90% lower than the ammonium production of cells maintained in a culture medium that is substantially free from choline and beta-alanine. In a further embodiment, the ammonium production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than ammonium production of cells maintained in a culture medium that is substantially free from choline and beta-alanine.

In one embodiment, the ammonium production of the cells is lower than the ammonium production of cells maintained in a culture medium that is substantially free of one or more of hypotaurine, Gamma-Aminobutyric Acid (GABA), and beta-alanine. In a further embodiment, the ammonium production of the cells is between about 5% and about 90% lower than the ammonium production of cells maintained in a culture medium that is substantially free from one or more of hypotaurine, Gamma-Aminobutyric Acid (GABA), and beta-alanine. In a further embodiment, the ammonium production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than ammonium production of cells maintained in a culture medium that is substantially free from one or more of hypotaurine, Gamma-Aminobutyric Acid (GABA), and beta-alanine.

In one embodiment, the ammonium concentration of the culture is between about 0.1 mM and about 20 mM. In a further embodiment, the ammonium concentration of the culture is between about 0.1 mM and about 15 mM, about 0.1 mM and about 14 mM, about 0.1 mM and about 13 mM, about 0.1 mM and about 12 mM, about 0.1 mM and about 11 mM, about 0.1 mM and about 10 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.5 mM and about 15 mM, about 0.5 mM and about 14 mM, about 0.5 mM and about 13 mM, about 0.5 mM and about 12 mM, about 0.5 mM and about 11 mM, about 0.5 mM and about 10 mM, about 0.5 mM and about 9 mM, about 0.5 mM and about 8 mM, about 0.5 mM and about 7 mM, about 0.5 mM and about 6 mM, about 0.5 mM and about 5 mM, about 0.5 mM and about 4 mM, about 0.5 mM and about 3 mM, about 0.5 mM and about 2 mM, about 0.5 mM and about 1 mM, about 1 mM and about 15 mM, about 1 mM and about 14 mM, about 1 mM and about 13 mM, about 1 mM and about 12 mM, about 1 mM and about 11 mM, about 1 mM and about 10 mM, about 1 mM and about 9 mM, about 1 mM and about 8 mM, about 1 mM and about 7 mM, about 1 mM and about 6 mM, about 1 mM and about 5 mM, about 1 mM and about 4 mM, about 1 mM and about 3 mM, or about 1 mM and about 2 mM. In one embodiment, the ammonium concentration of the culture is less than about 20 mM, about 19 mM, about 18 mM, about 17 mM, about 16 mM, about 15 mM, about 14 mM, about 13 mM, about 12, mM, about 11 mM, about 10 mM, about 9 mM, about 8 mM, about 7 mM, about 6 mM, about 5 mM, about 4 mM, about 3 mM, about 2 mM, about 1 mM, or about 0.5 mM.

In one embodiment, the cell specific lactate production rate to the cell specific glucose uptake rate ratio (LPR/GUR ratio) of the cells is between about −0.5 and about 0.5. In a further embodiment, the LPR/GUR ratio of the cells is between about −0.4 and about 0.5, about −0.3 and about 0.5, about −0.2 and about 0.5, about −0.1 and about 0.5, about −0.5 and about 0.4, about −0.5 and about 0.3, about −0.5 and about 0.2, about −0.5 and about 0.1, about −0.4 and about 0.4, about −0.3 and about 0.3, about −0.2 and about 0.2, about −0.1 and about 0.1, about −0.1 and about 0.5, about −0.2 and about 0.1, or about −0.3 and about 0.1.

In one embodiment, the cells are selected from the group consisting of CHO cells (including CHO-S and CHO-Ki), HEK cells, NS0 cells, PER.C6 cells, HeLa cells, and MDCK cells. In one embodiment, the cells are CHO cells. In another embodiment, the cells are HEK cells. In yet another embodiment, the cells are hybridoma cells. In one embodiment, the cells have been adapted to grow in serum free medium, animal protein free medium or chemically defined medium. In one embodiment, the cells have been genetically modified to alter their innate glycosylation pathways. In one embodiment, the cells have been genetically modified to increase their life-span in culture.

In one embodiment, the polypeptide of interest is selected from the group consisting of: an antibody, a Transforming Growth Factor (TGF) beta superfamily signaling molecule, an Fc fusion protein, interferon beta-1a, Lingo, CD40L, and a clotting factor. In another embodiment, the polypeptide of interest is interferon beta-1a. In another embodiment, the polypeptide is CD40L.

In one embodiment, the polypeptide of interest is an antibody. In one embodiment, the antibody is an IgA, IgD, IgE, IgG, or IgM. In one embodiment, the antibody is an IgG1, IgG2, IgG3, or IgG4. In one embodiment, the antibody is a full antibody. In one embodiment, the antibody is a chimeric antibody, humanized antibody or human antibody. In one embodiment, the antibody is a human IgG1 antibody. In one embodiment, the antibody is an anti-α4-integrin antibody. In another embodiment, the antibody is natalizumab. In another embodiment, the antibody is an anti-TWEAK antibody. In another embodiment, the antibody is anti-LINGO antibody. In another embodiment, the antibody is an anti-amyloid beta antibody. In one embodiment, the antibody is an anti-CD20 antibody. In another embodiment, the antibody is rituximab. In another embodiment, the antibody is obinutuzumab. In one embodiment, the antibody is an anti-IL2 antibody. In another embodiment, the antibody is daclizumab. In one embodiment, the antibody is an anti-αvβ6 integrin antibody. In one embodiment, the antibody is an anti-tau antibody.

In one embodiment, the TGF-beta superfamily signaling molecule is Neublastin.

In one embodiment, the clotting factor is a full-length clotting factor, a mature clotting factor, or a chimeric clotting factor.

In one embodiment, the total amount of polypeptide produced by the cells is higher than the total amount of polypeptide produced by cells maintained in a culture medium that is substantially free from choline and hypotaurine. In a further embodiment, the total amount of polypeptide produced by the cell is between about 5% and about 500% higher than the total amount of polypeptide produced by the cells maintained in a culture medium that is substantially free from choline and hypotaurine. In yet a further embodiment, the total amount of polypeptide produced by the cell is between about 5% and about 300% higher than the total amount of polypeptide produced by the cells maintained in a culture medium that is substantially free from choline and hypotaurine.

In one embodiment, the total amount of polypeptide produced by the cells is higher than the total amount of polypeptide produced by cells maintained in a culture medium that is substantially free from choline and Gamma-Aminobutyric Acid (GABA). In a further embodiment, the total amount of polypeptide produced by the cell is between about 5% and about 500% higher than the total amount of polypeptide produced by the cells maintained in a culture medium that is substantially free from choline and GABA. In yet a further embodiment, the total amount of polypeptide produced by the cell is between about 5% and about 300% higher than the total amount of polypeptide produced by the cells maintained in a culture medium that is substantially free from choline and GABA.

In one embodiment, the total amount of polypeptide produced by the cells is higher than the total amount of polypeptide produced by cells maintained in a culture medium that is substantially free from choline and beta-alanine. In a further embodiment, the total amount of polypeptide produced by the cell is between about 5% and about 500% higher than the total amount of polypeptide produced by the cells maintained in a culture medium that is substantially free from choline and beta-alanine. In yet a further embodiment, the total amount of polypeptide produced by the cell is between about 5% and about 300% higher than the total amount of polypeptide produced by the cells maintained in a culture medium that is substantially free from choline and beta-alanine.

In one embodiment, the total amount of polypeptide produced by the cells is higher than the total amount of polypeptide produced by cells maintained in a culture medium that is substantially free from one or more of choline, hypotaurine, Gamma-Aminobutyric Acid (GABA), and beta-alanine. In a further embodiment, the total amount of polypeptide produced by the cell is between about 5% and about 500% higher than the total amount of polypeptide produced by the cells maintained in a culture medium that is substantially free from one or more of hypotaurine, Gamma-Aminobutyric Acid (GABA), and beta-alanine. In yet a further embodiment, the total amount of polypeptide produced by the cell is between about 5% and about 300% higher than the total amount of polypeptide produced by the cells maintained in a culture medium that is substantially free from one or more of hypotaurine, Gamma-Aminobutyric Acid (GABA), and beta-alanine.

In one embodiment, the specific productivity of the cells is higher than the specific productivity of cells maintained in a culture medium that is substantially free of choline and hypotaurine. In a further embodiment, the specific productivity of the cells is between about 5% and about 500% higher than the specific productivity of cells maintained in a culture medium that is substantially free from choline and hypotaurine. In yet a further embodiment, the specific productivity of the cells is between about 5% and about 300% higher than the specific productivity of cells maintained in a culture medium that is substantially free from choline and hypotaurine.

In one embodiment, the specific productivity of the cells is higher than the specific productivity of cells maintained in a culture medium that is substantially free of choline and Gamma-Aminobutyric Acid (GABA). In a further embodiment, the specific productivity of the cells is between about 5% and about 500% higher than the specific productivity of cells maintained in a culture medium that is substantially free from choline and GABA. In yet a further embodiment, the specific productivity of the cells is between about 5% and about 300% higher than the specific productivity of cells maintained in a culture medium that is substantially free from choline and GABA.

In one embodiment, the specific productivity of the cells is higher than the specific productivity of cells maintained in a culture medium that is substantially free of choline and beta-alanine. In a further embodiment, the specific productivity of the cells is between about 5% and about 500% higher than the specific productivity of cells maintained in a culture medium that is substantially free from choline and beta-alanine. In yet a further embodiment, the specific productivity of the cells is between about 5% and about 300% higher than the specific productivity of cells maintained in a culture medium that is substantially free from choline and beta-alanine.

In one embodiment, the specific productivity of the cells is higher than the specific productivity of cells maintained in a culture medium that is substantially free of one or more of choline, hypotaurine, Gamma-Aminobutyric Acid (GABA), and beta-alanine. In a further embodiment, the specific productivity of the cells is between about 5% and about 500% higher than the specific productivity of cells maintained in a culture medium that is substantially free from one or more of hypotaurine, Gamma-Aminobutyric Acid (GABA), and beta-alanine. In yet a further embodiment, the specific productivity of the cells is between about 5% and about 300% higher than the specific productivity of cells maintained in a culture medium that is substantially free from one or more of hypotaurine, Gamma-Aminobutyric Acid (GABA), and beta-alanine.

In one embodiment, the culture is a perfusion culture. In another embodiment, the culture is a fed batch culture. In another embodiment, the culture is conducted in a shake flask. In yet another embodiment, the culture is conducted in a stirred-tank bioreactor. In one embodiment, the cell culture has a volume between about 500 liters and about 30,000 liters. In one embodiment, the medium is a serum free medium, animal protein free medium, or a chemically defined medium. In a preferred embodiment, the medium is a chemically defined medium.

In one embodiment, the hypotaurine, GABA, and/or beta-alanine is/are introduced into the culture medium as part of a feed medium. In one embodiment, the hypotaurine, GABA, and/or beta-alanine is/are introduced into the culture medium as one or more boli from a distinct stock solution. In one embodiment, the choline is introduced into the culture medium as part of a feed medium. In one embodiment, the choline is introduced into the culture medium as one or more boli from a distinct stock solution.

In one embodiment, the chimeric clotting factor comprises a Factor VIII polypeptide, a Factor VII polypeptide, a Factor IX polypeptide, a Von Willebrand Factor polypeptide, or any functional fragments thereof. In one embodiment, the chimeric clotting factor further comprises a heterologous moiety. In one embodiment, the heterologous moiety extends an in vivo half-life of the clotting factor. In one embodiment, the heterologous moiety is selected from the group consisting of albumin, albumin binding polypeptide, an FcRn binding partner, Fc, PAS, the β subunit of the C-terminal peptide (CTP) of human chorionic gonadotrophin, polyethylene glycol (PEG), hydroxyethyl starch (HES), albumin-binding small molecules, or combinations thereof. In one embodiment, the chimeric clotting factor is a monomer-dimer hybrid. In one embodiment, the Factor VII polypeptide comprises inactivated Factor VII, active Factor VII (FVIIa), or activatable Factor VII. In one embodiment, the Factor VIII polypeptide comprises full-length Factor VIII, mature Factor VIII, Factor VIII containing a partial or full deletion in B domain, or Factor VIII containing an insertion in one or more FVIII domains.

### BRIEF DESCRIPTION OF THE DRAWINGS/FIGURES

FIG. 1. In the presence of 9 mM choline chloride-containing feed medium, cells from Cell Line A exhibited higher growth (A), higher viability (B), lower ammonium accumulation (C), and higher titer (D) than cells cultured in the presence of a control feed medium containing 3 mM choline chloride.

FIG. 2. In the presence of 18 mM choline chloride-containing feed medium, cells from Cell Line B exhibited higher growth (A), higher viability (B), lower ammonium accumulation (C), and a slightly higher titer (D) than cells cultured in the presence of a control feed medium containing 3 mM choline chloride.

FIG. 3. In the presence of 8 mM hypotaurine-containing feed medium, cells from Cell Line B exhibited higher growth (A), higher viability (B), lower ammonium accumulation (C), and a higher titer (D) than cells cultured in the presence of a control feed medium containing 0 mM hypotaurine.

FIG. 4. In the presence of 18 mM choline chloride- and 4 mM hypotaurine-containing feed medium, cells from Cell Line B exhibited had little change in growth (A), viability (B), ammonium accumulation (C), and titer (D) compared to cells cultured in the presence of a control feed medium containing 3 mM choline chloride and 0 mM hypotaurine. In the presence of an 18 mM choline chloride- and 8 mM hypotaurine-containing feed medium, cells from Cell Line B exhibited higher growth (A), higher viability (B), and lower ammonium accumulation (C) than cells cultured in the presence of a control feed medium containing 3 mM choline and 0 mM hypotaurine. Feed regime 1 was used.

FIG. 5. In the presence of 18 mM choline chloride- and 8 mM hypotaurine-containing feed medium, cells from Cell Line B exhibited higher growth (A), higher viability (B), lower ammonium accumulation (C), lower lactate accumulation (D), and lower osmolality accumulation (E) than cells cultured in the presence of a control feed medium containing 3 mM choline chloride and 0 mM hypotaurine. The healthier culture and increased feeding regime allowed titers to be increased to >8 g/L on Day 16 (D). Feed regime 2 was used.

FIG. 6. Cell culture performance when 8 mM taurine was added to feed medium as compared to cell culture performance when 8 mM hypotaurine was added to feed medium. Taurine was not able to serve as a replacement for hypotaurine for growth (A), viability (B), ammonium concentration (C) or titer (D).

FIG. 7. Comparison of 3 mM cysteamine/18 mM choline chloride in feed medium, 1.3 mM glutathione/18 mM choline chloride in feed medium, and 8 mM hypotaurine and 18 mM choline chloride in feed medium. Cysteamine and glutathione were not able to serve as replacements for hypotaurine for growth (A), viability (B), ammonium concentration (C) or titer (D).

FIG. 8. Comparison of 18 mM choline chloride in feed medium with the combination of 18 mM choline chloride and 8 mM hypotaurine in feed medium. The control feed contained 3 mM choline chloride and 0 mM hypotaurine. The combination of hypotaurine and choline in the feed medium was associated with higher growth (A), higher viability (B), and lower ammonium accumulation (C) than the control feed medium or the choline-alone feed medium. Additionally, the titer (D) of the choline- and hypotaurine-containing feed medium experiment was higher.

FIG. 9. In the presence of 8 mM Gamma-Aminobutyric Acid (GABA)-containing feed medium, CHO cells exhibited higher viable cell density (cell growth) (A), higher viability (B), and lower lactate accumulation (C) than CHO cells cultured in the presence of a control feed medium that was not supplemented with GABA. GABA supplementation delays the onset of a high lactate phenotype by 2 days compared to control (C) and prolongs culture duration (A,B,C).

### DETAILED DESCRIPTION OF THE INVENTION

### I. Definitions

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever embodiments are described with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments of the disclosure, which can be had by reference to the specification as a whole. Accordingly, the terms defined immediately below are more fully defined by reference to the specification in its entirety.

The terms “polypeptide” or “protein” as used herein refer to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. If a single polypeptide is the discrete functioning unit and does require permanent physical association with other polypeptides in order to form the discrete functioning unit, the terms “polypeptide” and “protein” as used herein are used interchangeably. If the discrete functional unit is comprised of more than one polypeptide that physically associate with one another, the term “protein” as used herein refers to the multiple polypeptides that are physically coupled and function together as the discrete unit.

The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, monovalent or monospecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

“Recombinantly expressed” and “recombinant” as used herein refer to a polypeptide expressed from a host cell that has been genetically engineered to express that polypeptide. The recombinantly expressed polypeptide can be identical or similar to a polypeptide that is normally expressed in the mammalian host cell. The recombinantly expressed polypeptide can also be foreign to the host cell, i.e. heterologous to peptides normally expressed in the mammalian host cell. Alternatively, the recombinantly expressed polypeptide can be chimeric in that portions of the polypeptide contain amino acid sequences that are identical or similar to a polypeptide normally expressed in the mammalian host cell, while other portions are foreign to the host cell. In certain embodiments, the recombinant polypeptide comprises an antibody or fragments thereof. As used herein, the terms “recombinantly expressed polypeptide” and “recombinant polypeptide” also encompasses an antibody produced by a hybridoma.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “basal media formulation” or “basal media” as used herein refers to any cell culture media used to culture cells that has not been modified either by supplementation, or by selective removal of a certain component.

As used herein, the terms “additive” or “supplement” refer to any supplementation made to a basal medium to achieve the goals described in this disclosure. An “additive” or “supplement” can include a single substance, e.g., hypotaurine, Gamma-Aminobutyric Acid (GABA), beta-alanine, or choline or can include multiple substances, e.g., hypotaurine and choline; GABA and choline; beta-alanine and choline; choline and one or more of hypotaurine, GABA, and beta-alanine; choline and two or more of hypotaurine, GABA, and beta-alanine; two or more of hypotaurine, GABA, and beta-alanine. An “additive” or “supplement” can comprise a substance selected from the group consisting of hypotaurine, Gamma-Aminobutyric Acid (GABA), and beta-alanine. The terms “additive” or “supplement” refer to all of the components added, even though they need not be added at the same time, and they need not be added in the same way. For example, one or more components of an “additive” or “supplement” can be added as a single bolus or two or more boli from a stock solution, while other components of the same “additive” or “supplement” can be added as part of a feed medium. In addition, any one or more components of an “additive” or “supplement” can be present in the basal medium from the beginning of the cell culture.

The terms “culture”, “cell culture” and “eukaryotic cell culture” as used herein refer to a eukaryotic cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the mammalian cell population and the medium in which the population is suspended.

The terms “media”, “medium”, “feed”, “cell culture medium”, “culture medium”, “tissue culture medium”, “tissue culture media”, “growth medium”, and “feed medium” as used herein refer to a solution containing nutrients which nourish growing cultured eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant glycoproteins or proteins, for example, but not limited to, hormones, cytokines, interleukins and other signaling molecules. A “complete feed”, “complete media”, or “complete medium” as used herein refers to a media comprising at least all nutritional elements necessary to for culturing the reference organism including, e.g., glucose, amino acids, vitamins, and metals. A complete feed includes, e.g., CF2b (Huang et al., Biotechnology Progress 26(5):1400-1410 (2010)) and CM3 media (Gilbert et al., Biotechnology Progress 29:1519-1527 (2013)). One of skill in the art may readily determine by known techniques what constitutes “all necessary” “nutritional elements” for culturing a reference organism.

The cell culture medium is generally “serum free” when the medium is essentially free of serum, or fractions thereof, from any mammalian source (e.g. fetal bovine serum (FBS)). By “essentially free” is meant that the cell culture medium comprises between about 0-5% serum, preferably between about 0-1% serum, and most preferably between about 0-0.1% serum. Advantageously, serum-free “defined” medium can be used, wherein the identity and concentration of each of the components in the medium is known (i.e., an undefined component such as bovine pituitary extract (BPE) is not present in the culture medium).

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

The term “cell density” as used herein refers to that number of cells present in a given volume of medium.

The term “batch culture” as used herein refers to a method of culturing cells in which all the components that will ultimately be used in culturing the cells, including the medium (see definition of “medium”) as well as the cells themselves, are provided at the beginning of the culturing process. A batch culture is typically stopped at some point and the cells and/or components in the medium are harvested and optionally purified.

The term “fed-batch culture” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified. See Kshirsagar et al., Biotechnology Bioengineering 109:2523-2532 (2012).

The term “perfusion culture” as used herein refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified.

The term “bioreactor” as used herein refers to any vessel used for the growth of a mammalian cell culture. The bioreactor can be of any size so long as it is useful for the culturing of mammalian cells. Typically, the bioreactor will be at least 1 liter and can be 10, 50, 100, 250, 500, 1000, 2000, 2500, 3000, 5000, 8000, 10,000, 12,0000, 15,000, 20,000, 30,000 liters or more, or any volume in between. For example, a bioreactor will be 10 to 5,000 liters, 10 to 10,000 liters, 10 to 15,000 liters, 10 to 20,000 liters, 10 to 30,000 liters, 50 to 5,000 liters, 50 to 10,000 liters, 50 to 15,000 liters, 50 to 20,000 liters, 50 to 30,000 liters, 1,000 to 5,000 liters, or 1,000 to 3,000 liters. A bioreactor can be a stirred-tank bioreactor or a shake flask. The internal conditions of the bioreactor, for example, but not limited to pH and temperature, are typically controlled during the culturing period. The bioreactor can be composed of any material that is suitable for holding mammalian cell cultures suspended in media under the culture conditions of the present invention, including glass, plastic or metal. The term “production bioreactor” as used herein refers to the final bioreactor used in the production of the glycoprotein or protein of interest. The volume of the large-scale cell culture production bioreactor is typically at least 500 liters and can be 1000, 2000, 2500, 5000, 8000, 10,000, 12,0000, 15,000 liters or more, or any volume in between. For example, the large scale cell culture reactor will be between about 500 liters and about 30,000 liters, about 500 liters and about 20,000 liters, about 500 liters and about 10,000 liters, about 500 liters and about 5,000 liters, about 1,000 liters and about 30,000 liters, about 2,000 liters and about 30,000 liters, about 3,000 liters and about 30,000 liters, about 5,000 liters and about 30,000 liters, or about 10,000 liters and about 30,000 liters, or a large scale cell culture reactor will be at least about 500 liters, at least about 1,000 liters, at least about 2,000 liters, at least about 3,000 liters, at least about 5,000 liters, at least about 10,000 liters, at least about 15,000 liters, or at least about 20,000 liters. One of ordinary skill in the art will be aware of and will be able to choose suitable bioreactors for use in practicing the present invention.

The term “stirred-tank bioreactor” as used herein refers to any vessel used for the growth of a mammalian cell culture that has an impeller.

The term “shake flask” as used herein refers to any vessel used for the growth of a mammalian cell culture that does not have an impeller.

The term “hybridoma” as used herein refers to a cell created by fusion of an immortalized cell derived from an immunologic source and an antibody-producing cell. The resulting hybridoma is an immortalized cell that produces antibodies. The individual cells used to create the hybridoma can be from any mammalian source, including, but not limited to, rat, pig, rabbit, sheep, goat, and human. The term also encompasses trioma cell lines, which result when progeny of heterohybrid myeloma fusions, which are the product of a fusion between human cells and a murine myeloma cell line, are subsequently fused with a plasma cell. Furthermore, the term is meant to include any immortalized hybrid cell line that produces antibodies such as, for example, quadromas (See, e.g., Milstein et al., Nature, 537:3053 (1983)).

The term “osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of water (1 mOsm/kg H₂O at 38° C. is equivalent to an osmotic pressure of 19 mm Hg). “Osmolarity” refers to the number of solute particles dissolved in 1 liter of solution. Solutes which can be added to the culture medium so as to increase the osmolality thereof include proteins, peptides, amino acids, non-metabolized polymers, vitamins, ions, salts, sugars, metabolites, organic acids, lipids, etc. In the preferred embodiment, the concentration of amino acids and NaCl in the culture medium is increased in order to achieve the desired osmolality ranges set forth herein. When used herein, the abbreviation “mOsm” means “milliosmoles/kg H₂O”.

The term “titer” as used herein refers to the total amount of recombinantly expressed glycoprotein or protein produced by a cell culture divided by a given amount of medium volume. Titer is typically expressed in units of milligrams of glycoprotein or protein per milliliter of medium or in units of grams of glycoprotein or protein per liter of medium.

The term “waste byproduct” or “waste product” includes any metabolic waste product of cell growth or maintenance that inhibits growth or protein-production. Examples of waste byproducts include, but are not limited to, ammonium and lactate.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., cellular viability). The difference between said two values is, for example, less than about 50%, less than about 40%, less than about 30%, less than about 20%, and/or less than about 10% as a function of the reference/comparator value.

The phrase “substantially reduced,” or “substantially different,” as used herein with regard to amounts or numerical values (and not as reference to the chemical process of reduction), denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., cellular viability). The difference between said two values is, for example, greater than about 10%, greater than about 20%, greater than about 30%, greater than about 40%, and/or greater than about 50% as a function of the value for the reference/comparator molecule.

### II. Cell Culture Medium and Methods of Using Thereof

Achievement of a robust, scalable production process includes more than increasing the product titer while maintaining high product quality. The process must also predictably require the main carbohydrate source to remain constant, such that the feeding strategy does not need to change across scales. As many processes use glucose as the main carbohydrate, and have lactate and ammonium as the main byproducts, the time course of these three critical chemicals should also scale.

One of the primary barriers to achieving mammalian fed-batch cell cultures that are both long and productive is the accumulation of growth- and protein production-inhibitory metabolic waste byproducts, such as ammonium and lactate. One popular method for reducing ammonium is to remove glutamine from the cell culture media. See PCT Publ. No. WO 02/101019. However, removal of glutamine is not an option in many instances because not all biopharmaceutical cell lines are capable of synthesizing glutamine de novo.

The present invention is based on the recognition that cell culture media supplemented with hypotaurine, GABA, and/or beta-alanine or a combination of hypotaurine, GABA, and/or beta-alanine with choline reduces the accumulation of waste byproducts, such as ammonium and lactate. Further, the present invention is based on the recognition that cell culture media supplemented with hypotaurine, GABA, and/or beta-alanine or a combination of hypotaurine, GABA, and/or beta-alanine with choline does not adversely affect cell growth or viability. Instead, cell culture media supplemented with hypotaurine, GABA, and/or beta-alanine or a combination of hypotaurine, GABA, and/or beta-alanine with choline lead to increases in cell growth and viability and increases in the amount of polypeptides produced in eukaryotic cell cultures.

Provided herein are methods to culture mammalian cells engineered to express a polypeptide of interest. Specifically this disclosure provides methods for controlling growth, viability and waste products by a mammalian cell expressing the polypeptide of interest by supplementing a tissue culture medium in which the cells are growing and/or producing the polypeptide of interest with an additive, or culturing eukaryotic cells engineered to express a polypeptide of interest in a tissue culture medium which has been supplemented with such an additive. In certain embodiments, polypeptides produced by the methods provided are recovered. In certain embodiments, the cell culture medium contains hypotaurine. In certain embodiments, the cell culture medium contains hypotaurine and choline. In certain embodiments, the cell culture medium contains GABA. In certain embodiments, the cell culture medium contains GABA and choline. In certain embodiments, the cell culture medium contains beta-alanine. In certain embodiments, the cell culture medium contains beta-alanine and choline. In certain embodiments, the cell culture medium contains beta-alanine and hypotaurine. In certain embodiments, the cell culture medium contains hypotaurine and GABA. In certain embodiments, the cell culture medium contains two or more of hypotaurine, GABA, and beta-alanine. In certain embodiments, the cell culture medium contains two or more of hypotaurine, GABA, and beta-alanine with choline.

In one embodiment, the feed medium comprises hypotaurine, GABA, or beta-alanine in an amount sufficient to achieve a hypotaurine, GABA, or beta-alanine, respectively, concentration in the culture of between about 0.1 mM and about 500 mM, between about 0.1 mM and about 400 mM, between about 0.1 mM and about 300 mM, between about 0.1 mM and about 200 mM, between about 0.1 mM and about 100 mM, between about 0.1 mM and about 50 mM, between about 0.1 mM and about 25 mM, between about 1 mM and about 25 mM, between about 10 mM and about 500 mM, between about 20 mM and about 500 mM, between about 50 mM and about 500 mM, between about 100 mM and about 500 mM, between about 200 mM and about 500 mM, between about 10 mM and about 100 mM, between about 5 mM and about 70 mM, between about 8 mM and about 65 mM, between about 50 mM and about 200 mM, between about 100 mM and about 400 mM, between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 2 M and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM. In one embodiment, the cell culture medium comprises between about 0.1 mM and about 500 mM hypotaurine, GABA, or beta-alanine. In another embodiment, the feed medium comprises hypotaurine, GABA, or beta-alanine in an amount sufficient to achieve a hypotaurine, GABA, or beta-alanine, respectively, concentration in the culture of about 0.1 mM, about 0.5 mM, about 1 mM, about 2 mM, about 3 mM, about 4 mM, about 5 mM, about 6 mM, about 7 mM, about 8 mM, about 9 mM, about 10 mM, about 20 mM, about 30 mM, about 40 mM, about 50 mM, about 60 mM, about 70 mM, about 80 mM, about 90 mM, about 100 mM, about 200 mM, about 300 mM, about 400 mM, or about 500 mM. A skilled artisan readily understands that the absolute amount of hypotaurine, GABA, or beta-alanine supplemented by a feed medium to a cell culture can be calculated from the volume of feed medium added to the culture and the hypotaurine, GABA, or beta-alanine, respectively, concentration of the feed medium. In some embodiments, the hypotaurine, GABA, or beta-alanine is introduced into the culture medium as part of a feed medium. In some embodiments, the culture is supplemented with a feed medium comprising a sufficient amount of hypotaurine, GABA, or beta-alanine to achieve a hypotaurine, GABA, or beta-alanine, respectively, concentration in the culture of between about 0.1 mM and about 500 mM. In some embodiments, the hypotaurine, GABA, or beta-alanine is introduced into the culture medium as one or more boli from a distinct stock solution.

In one embodiment, a medium according to the present invention comprises a mixture of hypotaurine, GABA, and/or beta-alanine with choline. A feed medium can comprise choline in an amount sufficient to achieve a choline concentration in the culture of between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.1 mM and about 0.5 mM, about 0.5 mM and about 10 mM, about 1 mM and about 10 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM. In some embodiments, a feed medium described herein comprises a sufficient amount of choline to achieve a choline concentration in the culture of about 0.1 mM, about 0.2 mM, about 0.5 mM, about 1 mM, about 2 mM, about 3 mM, about 4 mM, about 5 mM, about 6 mM, about 7 mM, about 8 mM, about 9 mM, or about 10 mM. A skilled artisan readily understands that the absolute amount of choline supplemented by a feed medium to a cell culture can be calculated from the volume of feed medium added to the culture and the choline concentration of the feed medium. In some embodiments, the choline is introduced into the culture medium as one or more boli from a distinct stock solution. In some embodiments, the choline is introduced into the culture medium as part of a feed medium.

In one embodiment, the cells are maintained in a cell culture medium containing hypotaurine, GABA, and/or beta-alanine or a combination of choline with hypotaurine, GABA, and/or beta-alanine for between about 1 day and about 20 days. In another embodiment, the cells are maintained in a cell culture medium containing hypotaurine, GABA, and/or beta-alanine for between about 1 day and about 20 days, about 1 day and about 15 days, about 1 day and about 10 days, about 1 day and about 9 days, about 1 day and about 8 days, or about 1 day and about 7 days. In further embodiments, the cells are maintained in a cell culture medium containing hypotaurine, GABA, and/or beta-alanine or a combination of hypotaurine, GABA, and/or beta-alanine with choline for at least about 1 day, at least about 2 days, at least about 3 days, at least about 4 days, at least about 5 days, at least about 6 days, at least about 7 days, at least about 8 days, at least about 9 days, at least about 10 days, at least about 15 days, or at least about 20 days. In one embodiment, the concentration of hypotaurine, GABA, and/or beta-alanine in the cell culture medium is between 0.1 mM and 500 mM.

In one embodiment, a medium described herein is a serum-free medium, animal protein-free medium or a chemically-defined medium. In a specific embodiment, a medium described herein is a chemically-defined medium.

The present invention further provides a cell culture composition comprising a medium described herein and cells.

In one embodiment, a cell culture composition produced by the provided methods can be a batch culture, fed-batch culture, a perfusion culture, a shake flask, and/or a bioreactor. In a specific embodiment, a cell culture composition of the invention is a fed batch culture. In one embodiment, cells expressing a polypeptide of interest are cultured in basal medium to which the additive is introduced as a bolus, or two or more boli, from a stock solution. In another embodiment, the additive is introduced as a component of a feed medium. In certain embodiments the cell culture comprises a growth phase and a protein production phase, and the additive is introduced into the culture medium before, or at the same time as, or at some point after the initiation of the protein production phase. In one embodiment the additive is hypotaurine. In other embodiments, the additives include hypotaurine and choline. In one embodiment the additive is Gamma-Aminobutyric Acid (GABA). In other embodiments, the additives include GABA and choline. In one embodiment the additive is beta-alanine. In other embodiments, the additives include beta-alanine and choline. In other embodiments, the additives include beta-alanine and hypotaurine. In other embodiments, the additives include hypotaurine and GABA. In other embodiments, the additives include beta-alanine and choline. In one embodiment the additive is selected from the group consisting of hypotaurine, GABA, and beta-alanine. In other embodiments, the additives include choline and a supplement selected from the group consisting of hypotaurine, GABA, and beta-alanine. In one embodiment the additive is two or more of hypotaurine, GABA, and beta-alanine. In other embodiments, the additives include two or more of hypotaurine, GABA, and beta-alanine with choline.

In one embodiment, a cell culture composition produced by the provided methods comprises eukaryotic cells. In another embodiment, a cell culture composition produced by the provided methods comprises mammalian cells selected from the group consisting of CHO cells (including CHO-S and CHO-K1 cells), HEK cells, NS0 cells, PER.C6 cells, 293 cells, HeLa cells, and MDCK cells. In a specific embodiment, a cell culture composition described herein comprises CHO cells. In another specific embodiment, a cell culture composition described herein comprises HEK cells. In another specific embodiment, a cell culture composition described herein comprises hybridoma cells.

A cell culture composition produced by the provided methods can comprise cells that have been adapted to grow in serum free medium, animal protein free medium or chemically defined medium. Or it can comprise cells that have been genetically modified to increase their life-span in culture.

The present invention provides a method of culturing cells, comprising contacting the cells with a medium disclosed herein, supplementing the medium as described above, or culturing cells in a medium supplemented as described above.

Cell cultures can be cultured in a batch culture, fed batch culture, a perfusion culture, shake-flask culture, or a bioreactor. In one embodiment, a cell culture according to a method of the present invention is a batch culture. In another embodiment, a cell culture according to a method of the present invention is a fed batch culture. In a further embodiment, a cell culture according to a method of the present invention is a perfusion culture. In certain embodiments the cell culture is maintained in a shake flask. In certain embodiments the cell culture is maintained in a bioreactor. In one embodiment, the culture is conducted in a stirred-tank bioreactor. In certain embodiments, the cell culture has a volume between about 500 liters and about 30,000 liters.

In one embodiment, a cell culture according to a method of the present invention is a serum-free culture. In another embodiment, a cell culture according to a method of the present invention is a chemically defined culture. In a further embodiment, a cell culture according to a method of the present invention is an animal protein free culture.

In one embodiment, a cell culture produced by the provided methods is contacted with a medium described herein during the growth phase of the culture. In another embodiment, a cell culture is contacted with a medium described herein during the production phase of the culture. In one embodiment, the cell culture is supplemented with a feed medium containing hypotaurine. In one embodiment, the cells culture is supplemented with a feed medium containing both hypotaurine and choline. In one embodiment the cell culture is supplemented with a feed medium containing Gamma-Aminobutyric Acid (GABA). In other embodiments, the cell culture is supplemented with a feed medium containing GABA and choline. In one embodiment the cell culture is supplemented with a feed medium containing beta-alanine. In other embodiments, the cell culture is supplemented with a feed medium containing beta-alanine and choline. In other embodiments, the cell culture is supplemented with a feed medium containing beta-alanine and hypotaurine. In other embodiments, the cell culture is supplemented with a feed medium containing beta-alanine and hypotaurine. In other embodiments, the cell culture is supplemented with a feed medium containing GABA and hypotaurine. In one embodiment the cell culture is supplemented with a feed medium containing two or more of hypotaurine, GABA, and beta-alanine. In other embodiments, the cell culture is supplemented with a feed medium containing choline and two or more of hypotaurine, GABA, and beta-alanine.

In one embodiment, the culture is supplemented with the feed medium between about 1 and about 20 times. In another embodiment, a culture is supplemented with the feed medium between about 1 and about 20 times, between about 1 and about 15 times, or between about 1 and about 10 times. In a further embodiment, a culture is supplemented with the feed medium at least once, at least twice, at least three times, at least four times, at least five times, at least 6 times, at least 7 times, at least 8 times, at least 9 times, at least 10 times, at least 1 times, at least 12 times, at least 13 times, at least 14 times, at least 15 times, or at least 20 times.

A culture produced by the provided methods can be contacted with a feed medium described herein at regular intervals. In one embodiment, the regular interval is about once a day, about once every two days, about once every three days, about once every 4 days, or about once every 5 days.

A culture produced by the provided methods can be contacted with a feed medium described herein on an as needed basis based on the metabolic status of the culture. In one embodiment, a metabolic marker of a fed batch culture is measured prior to supplementing the culture with a feed medium described herein. In one embodiment, the metabolic marker is selected from the group consisting of: lactate concentration, ammonium concentration, alanine concentration, glutamine concentration, glutamate concentration, cell specific lactate production rate to the cell specific glucose uptake rate ratio (LPR/GUR ratio), and Rhodamine 123 specific cell fluorescence. In one embodiment, an LPR/GUR value of >0.1 indicates the need to supplement the culture with a feed medium described herein. In a further specific embodiment, a lactate concentration of >3 g/L indicates the need to supplement the culture with a feed medium described herein. In another embodiment, a culture according to the present invention is supplemented with a feed medium described herein when the LPR/GUR value of the culture is >0.1 or when the lactate concentration of the culture is >3 g/L.

In one embodiment, the cell specific lactate production rate to the cell specific glucose uptake rate ratio (LPR/GUR ratio) of the cells is between about −0.5 and about 0.5. In one embodiment, the LPR/GUR ratio of the cells is between about −0.4 and about 0.5, about −0.3 and about 0.5, about −0.2 and about 0.5, about −0.1 and about 0.5, about −0.5 and about 0.4, about −0.5 and about 0.3, about −0.5 and about 0.2, about −0.5 and about 0.1, about −0.4 and about 0.4, about −0.3 and about 0.3, about −0.2 and about 0.2, about −0.1 and about 0.1, about −0.1 and about 0.5, about −0.2 and about 0.1, or about −0.3 and about 0.1.

In one embodiment, the osmolality is reduced. In some embodiments, the osmolality is less than about 600 mOsm, less than about 500 mOsm, less than about 450 mOsm, less than about 400 mOsm, less than about 350 mOsm, less than about 300 mOsm, less than about 250 mOsm, or less than about 200 mOsm.

In one embodiment, supplementation of the media by either hypotaurine; GABA;

beta-alanine; hypotaurine and choline; GABA and choline; beta-alanine and choline; GABA and hypotaurine; beta-alanine and hypotaurine; GABA and beta-alanine; two or more of hypotaurine, GABA, beta-alanine, and choline; or choline and one or more of hypotaurine, GABA, and beta-alanine results in a reduction in a waste byproduct of the cultured mammalian cells. In one embodiment, the waste byproduct is lactate. In one embodiment, the waste product is ammonium. In another embodiment, both lactate and ammonium are reduced.

In one embodiment, the lactate production of the cells is lower than the lactate production of cells maintained in a culture medium that is substantially free from choline and hypotaurine. In one embodiment, the lactate production of the cells is lower than the lactate production of cells maintained in a culture medium that is substantially free from choline and GABA. In one embodiment, the lactate production of the cells is lower than the lactate production of cells maintained in a culture medium that is substantially free from choline and beta-alanine. In one embodiment, the lactate production of the cells is lower than the lactate production of cells maintained in a culture medium that is substantially free from hypotaurine and GABA. In one embodiment, the lactate production of the cells is lower than the lactate production of cells maintained in a culture medium that is substantially free from hypotaurine and beta-alanine. In one embodiment, the lactate production of the cells is lower than the lactate production of cells maintained in a culture medium that is substantially free from beta-alanine and GABA. In one embodiment, the lactate production of the cells is between about 5% and about 80%, between about 5% and about 70%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, or between about 50% and about 90% lower than the lactate production of cells maintained in a culture medium that is substantially free of choline and hypotaurine. In one embodiment, the lactate production of the cells is between about 5% and about 95% lower than the lactate production of cells maintained in a culture medium that is substantially free from choline and hypotaurine. In one embodiment, the lactate concentration of the culture is between about 0.1 g/L and about 10 g/L, between about 0.1 g/L and about 9 g/L, between about 0.1 g/L and about 8 g/L, between about 10 g/L and about 7 g/L, between about 10 g/L and about 6 g/L, between about 10 g/L and about 5 g/L, between about 0.1 g/L and about 4 g/L, between about 0.1 g/L and about 3 g/L, between about 0.1 g/L and about 2 g/L, between about 0.1 g/L and about 2 g/L, between about 0.1 g/L and about 1 g/L, between about 0.1 g/L and about 0.5 g/L, between about 0.5 g/L and about 5 g/L, between about 1 g/L and about 5 g/L, between about 2 g/L and about 5 g/L, between about 3 g/L and about 5 g/L, or between about 4 g/L and about 5 g/L. In one embodiment, the lactate concentration of the culture is between about 0.1 g/L and about 10 g/L. In one embodiment, the lactate concentration of the culture is less than about 10 g/L, about 9 g/L, about 8 g/L, about 7 g/L, about 6 g/L, about 5 g/L, about 4 g/L, about 3 g/L, about 2 g/L, about 1 g/L, or about 0.5 g/L.

In one embodiment, the lactate production of the cells is between about 5% and about 80%, between about 5% and about 70%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, or between about 50% and about 90% lower than the lactate production of cells maintained in a culture medium that is substantially free of choline and Gamma-Aminobutyric Acid (GABA). In one embodiment, the lactate production of the cells is between about 5% and about 95% lower than the lactate production of cells maintained in a culture medium that is substantially free from choline and GABA. In one embodiment, the lactate concentration of the culture is between about 0.1 g/L and about 10 g/L, between about 0.1 g/L and about 9 g/L, between about 0.1 g/L and about 8 g/L, between about 10 g/L and about 7 g/L, between about 10 g/L and about 6 g/L, between about 10 g/L and about 5 g/L, between about 0.1 g/L and about 4 g/L, between about 0.1 g/L and about 3 g/L, between about 0.1 g/L and about 2 g/L, between about 0.1 g/L and about 2 g/L, between about 0.1 g/L and about 1 g/L, between about 0.1 g/L and about 0.5 g/L, between about 0.5 g/L and about 5 g/L, between about 1 g/L and about 5 g/L, between about 2 g/L and about 5 g/L, between about 3 g/L and about 5 g/L, or between about 4 g/L and about 5 g/L. In one embodiment, the lactate concentration of the culture is between about 0.1 g/L and about 10 g/L. In one embodiment, the lactate concentration of the culture is less than about 10 g/L, about 9 g/L, about 8 g/L, about 7 g/L, about 6 g/L, about 5 g/L, about 4 g/L, about 3 g/L, about 2 g/L, about 1 g/L, or about 0.5 g/L.

In one embodiment, the lactate production of the cells is between about 5% and about 80%, between about 5% and about 70%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, or between about 50% and about 90% lower than the lactate production of cells maintained in a culture medium that is substantially free of choline and beta-alanine. In one embodiment, the lactate production of the cells is between about 5% and about 95% lower than the lactate production of cells maintained in a culture medium that is substantially free from choline and beta-alanine. In one embodiment, the lactate concentration of the culture is between about 0.1 g/L and about 10 g/L, between about 0.1 g/L and about 9 g/L, between about 0.1 g/L and about 8 g/L, between about 10 g/L and about 7 g/L, between about 10 g/L and about 6 g/L, between about 10 g/L and about 5 g/L, between about 0.1 g/L and about 4 g/L, between about 0.1 g/L and about 3 g/L, between about 0.1 g/L and about 2 g/L, between about 0.1 g/L and about 2 g/L, between about 0.1 g/L and about 1 g/L, between about 0.1 g/L and about 0.5 g/L, between about 0.5 g/L and about 5 g/L, between about 1 g/L and about 5 g/L, between about 2 g/L and about 5 g/L, between about 3 g/L and about 5 g/L, or between about 4 g/L and about 5 g/L. In one embodiment, the lactate concentration of the culture is between about 0.1 g/L and about 10 g/L. In one embodiment, the lactate concentration of the culture is less than about 10 g/L, about 9 g/L, about 8 g/L, about 7 g/L, about 6 g/L, about 5 g/L, about 4 g/L, about 3 g/L, about 2 g/L, about 1 g/L, or about 0.5 g/L.

In one embodiment, the lactate production of the cells is between about 5% and about 80%, between about 5% and about 70%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, or between about 50% and about 90% lower than the lactate production of cells maintained in a culture medium that is substantially free of one or more of choline, hypotaurine, Gamma-Aminobutyric Acid (GABA), and beta-alanine. In one embodiment, the lactate production of the cells is between about 5% and about 95% lower than the lactate production of cells maintained in a culture medium that is substantially free of one or more of choline, hypotaurine, Gamma-Aminobutyric Acid (GABA), and beta-alanine. In one embodiment, the lactate concentration of the culture is between about 0.1 g/L and about 10 g/L, between about 0.1 g/L and about 9 g/L, between about 0.1 g/L and about 8 g/L, between about 10 g/L and about 7 g/L, between about 10 g/L and about 6 g/L, between about 10 g/L and about 5 g/L, between about 0.1 g/L and about 4 g/L, between about 0.1 g/L and about 3 g/L, between about 0.1 g/L and about 2 g/L, between about 0.1 g/L and about 2 g/L, between about 0.1 g/L and about 1 g/L, between about 0.1 g/L and about 0.5 g/L, between about 0.5 g/L and about 5 g/L, between about 1 g/L and about 5 g/L, between about 2 g/L and about 5 g/L, between about 3 g/L and about 5 g/L, or between about 4 g/L and about 5 g/L. In one embodiment, the lactate concentration of the culture is between about 0.1 g/L and about 10 g/L. In one embodiment, the lactate concentration of the culture is less than about 10 g/L, about 9 g/L, about 8 g/L, about 7 g/L, about 6 g/L, about 5 g/L, about 4 g/L, about 3 g/L, about 2 g/L, about 1 g/L, or about 0.5 g/L.

In one embodiment, the ammonium production of the cells is lower than the ammonium production of cells maintained in a culture medium that is substantially free of choline and hypotaurine. In one embodiment, the ammonium production of the cells is lower than the ammonium production of cells maintained in a culture medium that is substantially free from choline and GABA. In one embodiment, the ammonium production of the cells is lower than the ammonium production of cells maintained in a culture medium that is substantially free from choline and beta-alanine. In one embodiment, the ammonium production of the cells is lower than the lactate production of cells maintained in a culture medium that is substantially free from hypotaurine and GABA. In one embodiment, the ammonium production of the cells is lower than the ammonium production of cells maintained in a culture medium that is substantially free from hypotaurine and beta-alanine. In one embodiment, the ammonium production of the cells is lower than the ammonium production of cells maintained in a culture medium that is substantially free from beta-alanine and GABA. In one embodiment, the ammonium production of the cells is between about 5% and about 80%, between about 5% and about 70%, between about 5% and about 60%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, between about 50% and about 90%, between about 60% and about 90%, between about 70% and about 90%, or between about 80% and about 90% lower than ammonium production of cells maintained in a culture medium that is substantially free from choline and hypotaurine. In one embodiment, the ammonium production of cells is between about 5% and about 90% lower than the ammonium production of cells maintained in a culture medium that is substantially free from choline and hypotaurine. In one embodiment, the ammonium production of the cells is between about 5% and about 80%, between about 5% and about 70%, between about 5% and about 60%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, between about 50% and about 90%, between about 60% and about 90%, between about 70% and about 90%, or between about 80% and about 90% lower than ammonium production of cells maintained in a culture medium that is substantially free from choline and GABA. In one embodiment, the ammonium production of cells is between about 5% and about 90% lower than the ammonium production of cells maintained in a culture medium that is substantially free from choline and GABA. In one embodiment, the ammonium production of the cells is between about 5% and about 80%, between about 5% and about 70%, between about 5% and about 60%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, between about 50% and about 90%, between about 60% and about 90%, between about 70% and about 90%, or between about 80% and about 90% lower than ammonium production of cells maintained in a culture medium that is substantially free from choline and beta-alanine. In one embodiment, the ammonium production of cells is between about 5% and about 90% lower than the ammonium production of cells maintained in a culture medium that is substantially free from one or more of choline, hypotaurine, Gamma-Aminobutyric Acid (GABA), and beta-alanine. In one embodiment, the ammonium production of the cells is between about 5% and about 80%, between about 5% and about 70%, between about 5% and about 60%, between about 5% and about 50%, between about 5% and about 40%, between about 5% and about 30%, between about 5% and about 20%, between about 5% and about 10%, between about 10% and about 90%, between about 20% and about 90%, between about 30% and about 90%, between about 40% and about 90%, between about 50% and about 90%, between about 60% and about 90%, between about 70% and about 90%, or between about 80% and about 90% lower than ammonium production of cells maintained in a culture medium that is substantially free from one or more of choline, hypotaurine, Gamma-Aminobutyric Acid (GABA), and beta-alanine. In one embodiment, the ammonium production of cells is between about 5% and about 90% lower than the ammonium production of cells maintained in a culture medium that is substantially free from one or more of choline, hypotaurine, Gamma-Aminobutyric Acid (GABA), and beta-alanine. In one embodiment, the ammonium concentration of the culture is between about 0.1 mM and about 15 mM, about 0.1 mM and about 14 mM, about 0.1 mM and about 13 mM, about 0.1 mM and about 12 mM, about 0.1 mM and about 11 mM, about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.5 mM and about 15 mM, about 0.5 mM and about 14 mM, about 0.5 mM and about 13 mM, about 0.5 mM and about 12 mM, about 0.5 mM and about 11 mM, about 0.5 mM and about 10 mM, about 0.5 mM and about 9 mM, about 0.5 mM and about 8 mM, about 0.5 mM and about 7 mM, about 0.5 mM and about 6 mM, about 0.5 mM and about 5 mM, about 0.5 mM and about 4 mM, about 0.5 mM and about 3 mM, about 0.5 mM and about 2 mM, about 0.5 mM and about 1 mM, about 1 mM and about 15 mM, about 1 mM and about 14 mM, about 1 mM and about 13 mM, about 1 mM and about 12 mM, about 1 mM and about 11 mM, about 1 mM and about 10 mM, about 1 mM and about 9 mM, about 1 mM and about 8 mM, about 1 mM and about 7 mM, about 1 mM and about 6 mM, about 1 mM and about 5 mM, about 1 mM and about 4 mM, about 1 mM and about 3 mM, or about 1 mM and about 2 mM. In one embodiment, the ammonium concentration of the culture is between about 0.1 mM and about 20 mM. In one embodiment, the ammonium concentration of the culture is less than about 20 mM, about 19 mM, about 18 mM, about 17 mM, about 16 mM, about 15 mM, about 14 mM, about 13 mM, about 12, mM, about 11 mM, about 10 mM, about 9 mM, about 8 mM, about 7 mM, about 6 mM, about 5 mM, about 4 mM, about 3 mM, about 2 mM, about 1 mM, or about 0.5 mM.

In one embodiment, a medium described herein is a feed medium for a fed batch cell culture. A skilled artisan understands that a fed batch cell culture can be contacted with a feed medium more than once. In one embodiment, a fed batch cell culture is contacted with a medium described herein only once. In another embodiment, a fed batch cell culture is contacted with a medium described herein more than once, for example, at least twice, at least three times, at least four times, at least five times, at least six times, at least seven times, or at least ten times.

In accordance with the present invention, the total volume of feed medium added to a cell culture should optimally be kept to a minimal amount. For example, the total volume of the feed medium added to the cell culture can be 1, 2, 3, 4, 5, 6, 7, 8, 9, 10, 15, 20, 25, 30, 35, 40, 45 or 50% of the volume of the cell culture prior to adding the feed medium.

Cell cultures produced by the provided methods can be grown to achieve a particular cell density, depending on the needs of the practitioner and the requirement of the cells themselves, prior to being contacted with a medium described herein. In one embodiment, the cell culture is contacted with a medium described herein at a viable cell density of 1, 5, 10, 15, 20, 25, 30, 35, 40, 45, 50, 55, 60, 65, 70, 75, 80, 85, 90, 95 or 99 percent of maximal viable cell density. In a specific embodiment, the medium is a feed medium.

Cell cultures produced by the provided methods can be allowed to grow for a defined period of time before they are contacted with a medium described herein. In one embodiment, the cell culture is contacted with a medium described herein at day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the cell culture. In another embodiment, the cell culture is contacted with a medium described herein at week 1, 2, 3, 4, 5, 6, 7, or 8 of the cell culture. In a specific embodiment, the medium is a feed medium.

Cell cultures produced by the provided methods can be cultured in the production phase for a defined period of time. In one embodiment, the cell culture is contacted with a feed medium described herein at day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the production phase.

A culture produced by the provided methods can be maintained in production phase for between about 1 day and about 30 days. In one embodiment, a culture is maintained in production phase for between about 1 day and about 30 days, between about 1 day and about 25 days, between about 1 day and about 20 days, about 1 day and about 15 days, about 1 day and about 14 days, about 1 day and about 13 days, about 1 day and about 12 days, about 1 day and about 11 days, about 1 day and about 10 days, about 1 day and about 9 days, about 1 day and about 8 days, about 1 day and about 7 days, about 1 day and about 6 days, about 1 day and about 5 days, about 1 day and about 4 days, about 1 day and about 3 days, about 2 days and about 25 days, about 3 days and about 25 days, about 4 days and about 25 days, about 5 days and about 25 days, about 6 days and about 25 days, about 7 days and about 25 days, about 8 days and about 25 days, about 9 days and about 25 days, about 10 days and about 25 days, about 15 days and about 25 days, about 20 days and about 25 days, about 2 days and about 30 days, about 3 days and about 30 days, about 4 days and about 30 days, about 5 days and about 30 days, about 6 days and about 30 days, about 7 days and about 30 days, about 8 days and about 30 days, about 9 days and about 30 days, about 10 days and about 30 days, about 15 days and about 30 days, about 20 days and about 30 days, or about 25 days and about 30 days. In another embodiment, a culture is maintained in production phase for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 15 days, at least about 20 days, at least about 25 days, or at least about 30 days. In a further embodiment, a culture is maintained in production phase for about 1 day, about 2 days, about 3 days, about 4 days, about 5 days, about 6 days, about 7 days, about 8 days, about 9 days, about 10 days, about 11 days, about 12 days, about 15 days, about 20 days, about 25 days, or about 30 days.

In some embodiments, the viability of the cells is at least about 100%, at least about 99%, at least about 95%, at least about 90%, at least about 85%, at least about 80%, at least about 75%, at least about 70%, at least about 65%, at least about 60%, at least about 55%, at least about 50% or at least about 45% throughout the culture.

In one embodiment, the cells have been modified to express a polypeptide of interest. In some embodiments, the polypeptide is a recombinant polypeptide. In some embodiments, the polypeptide of interest is selected from the group consisting of: an antibody, a Transforming Growth Factor (TGF) beta superfamily signaling molecule, an Fc fusion protein, interferon beta-1a, Lingo, CD40L, and a clotting factor. In one embodiment, the polypeptide of interest is a TGF-beta superfamily signaling molecule. In one embodiment, TGF-beta superfamily signaling molecule is Neublastin. In another embodiment, the polypeptide of interest is CD40L.

In certain embodiments, the polypeptide of interest is an antibody or a fragment thereof. In a specific embodiment, the polypeptide is an antibody. In one embodiment the antibody is an anti-α4-integrin antibody. In yet another embodiment, the antibody is natalizumab. In another embodiment, the antibody is an anti-TWEAK antibody. In another embodiment, the antibody is anti-LINGO antibody. In another embodiment, the antibody is an anti-amyloid beta antibody. In one embodiment, the antibody is an anti-CD20 antibody. In another embodiment, the antibody is rituximab. In another embodiment, the antibody is obinutuzumab. In one embodiment, the antibody is an anti-IL2 antibody. In another embodiment, the antibody is daclizumab. In one embodiment, the antibody is an anti-αvβ6 integrin antibody. In one embodiment, the antibody is an anti-tau antibody. In another embodiment, the polypeptide is a blood clotting factor. The present invention further provides a method of producing a polypeptide of interest, comprising culturing cells engineered to express the polypeptide of interest in a culture comprising a medium described herein; and recovering or isolating the polypeptide of interest from the culture.

In one embodiment, the total amount of polypeptide produced by the cells is higher than the total amount of polypeptide produced by cells maintained in a culture medium that is substantially free from hypotaurine, GABA, beta-alanine, and choline. In one embodiment, the total amount of polypeptide produced by the cell is between about 5% and about 500% higher than the total amount of polypeptide produced by the cells maintained in a culture medium that is substantially free from hypotaurine, GABA, beta-alanine, and choline. In another embodiment, the total amount of polypeptide produced by the cell is between about 5% and about 300% higher than the total amount of polypeptide produced by the cells maintained in a culture medium that is substantially free from hypotaurine, GABA, beta-alanine, and choline. In one embodiment, the total amount of polypeptide produced by the cell is between about 5% and 400%, 5% and 300%, 5% and 200%, 5% and 150%, 5% and 100%, 5% and 99%, 5% and 95%, 5% and 90%, 5% and 80%, 5% and 75%, 5% and 50%, 5% and 25%, 10% and 500%, 20% and 500%, 50% and 500%, 100% and 500%, 200% and 500%, 300% and 500%, or 400% and 500% higher than the total amount of polypeptide produced by the cells maintained in a culture medium that is substantially free from hypotaurine, GABA, beta-alanine, and choline. In one embodiment, the total amount of polypeptide produced by the cell is about 5%, about 25%, about 50%, about 75%, about 90%, about 99%, about 100%, about 125%, about 150%, about 200%, about 250%, about 300%, about 350%, about 400%, about 450% or about 500% higher than the total amount of polypeptide produced by the cells maintained in a culture medium that is substantially free from hypotaurine, GABA, beta-alanine, and choline.

In a specific embodiment, a method of producing a polypeptide of interest according to the present invention produces a maximum polypeptide titer of at least about 0.05 g/L, at least about 0.1 g/L, at least about 0.25 g/L, at least about 0.5 g/L, at least about 0.75 g/L, at least about 1.0 g/L, at least about 1.5 g/L, at least about 2 g/liter, at least about 2.5 g/liter, at least about 3 g/liter, at least about 3.5 g/liter, at least about 4 g/liter, at least about 4.5 g/liter, at least about 5 g/liter, at least about 6 g/liter, at least about 7 g/liter, at least about 8 g/liter, at least about 9 g/liter, at least about 10 g/liter, at least about 11 g/liter, or at least about 12 g/liter. In another embodiment, the method according to the present invention produces a maximum polypeptide titer of between about 1 g/liter and about 10 g/liter, about 1 g/liter and about 12 g/liter, about 1.5 g/liter and about 10 g/liter, about 2 g/liter and about 10 g/liter, about 2.5 g/liter and about 10 g/liter, about 3 g/liter and about 10 g/liter, about 4 g/liter and about 10 g/liter, about 5 g/liter and about 10 g/liter, about 6 g/liter and about 10 g/liter, about 7 g/liter and about 10 g/liter, about 8 g/liter and about 10 g/liter, about 9 g/liter and about 10 g/liter, about 1 g/liter and about 9 g/liter, about 1 g/liter and about 8 g/liter, about 1 g/liter and about 7 g/liter, about 1 g/liter and about 6 g/liter, about 1 g/liter and about 5 g/liter, about 1 g/liter and about 4 g/liter, about 1 g/liter and about 3 g/liter, or about 1 g/liter and about 2 g/liter. In another embodiment, a method according to the present invention produces at least 2 times, three times, four times, five times, or ten times more protein or polypeptide.

In one embodiment, the specific productivity of the cells is higher than the specific productivity of cells maintained in a culture medium that is substantially free of hypotaurine, GABA, beta-alanine, and choline. In one embodiment, the specific productivity of the cells is between about 5% and about 500% higher than the specific productivity of cells maintained in a culture medium that is substantially free from hypotaurine, GABA, beta-alanine, and choline. In one embodiment, the specific productivity of the cells is between about 5% and about 300% higher than the specific productivity of cells maintained in a culture medium that is substantially free from hypotaurine, GABA, beta-alanine, and choline. In one embodiment, the specific productivity of the cells is between about 5% and 500%, 5% and 400%, 5% and 300%, 5% and 200%, 5% and 150%, 5% and 100%, 5% and 100%, 5% and 99%, 5% and 95%, 5% and 90%, 5% and 80%, 5% and 75%, 5% and 50%, or 5% and 25% higher than the specific productivity of cells maintained in a culture medium that is substantially free from hypotaurine, GABA, beta-alanine, and choline. In one embodiment, the specific productivity of the cells is about 5%, about 25%, about 50%, about 75%, about 90%, about 99%, about 100%, about 125%, about 150%, about 200%, about 250%, about 300%, about 350%, about 400%, about 450% or about 500% higher than the specific productivity of cells maintained in a culture medium that is substantially free from hypotaurine, GABA, beta-alanine, and choline.

The invention further provides a conditioned cell culture medium produced by a method described herein. In one embodiment, a conditioned cell culture medium produced according to the provided methods comprises a polypeptide of interest. In a specific embodiment, a conditioned cell culture medium according to the invention comprises a polypeptide of interest at a titer of at least about 2 g/liter, at least about 2.5 g/liter, at least about 3 g/liter, at least about 3.5 g/liter, at least about 4 g/liter, at least about 4.5 g/liter, at least about 5 g/liter, at least about 6 g/liter, at least about 7 g/liter, at least about 8 g/liter, at least about 9 g/liter, at least about 10 g/liter, at least about 11 g/liter, or at least about 12 g/liter, or a titer of between about 1 g/liter and about 10 g/liter, about 1 g/liter to about 12 g/liter, about 1.5 g/liter and about 10 g/liter, about 2 g/liter and about 10 g/liter, about 2.5 g/liter and about 10 g/liter, about 3 g/liter and about 10 g/liter, about 4 g/liter and about 10 g/liter, about 5 g/liter and about 10 g/liter, about 1 g/liter and about 5 g/liter, about 1 g/liter and about 4.5 g/liter, or about 1 g/liter and about 4 g/liter. In another embodiment, a conditioned cell culture medium according to the invention comprises a polypeptide of interest at a higher titer than the titer obtained without the use of a medium described herein. In a specific embodiment, the protein or polypeptide is an antibody.

Polypeptides

Any polypeptide that is expressible in a host cell can be produced in accordance with the present invention. The polypeptide can be expressed from a gene that is endogenous to the host cell, or from a gene that is introduced into the host cell through genetic engineering. The polypeptide can be one that occurs in nature, or can alternatively have a sequence that was engineered or selected by the hand of man. An engineered polypeptide can be assembled from other polypeptide segments that individually occur in nature, or can include one or more segments that are not naturally occurring.

Antibodies

Given the large number of antibodies currently in use or under investigation as pharmaceutical or other commercial agents, production of antibodies is of particular interest in accordance with the present invention. Antibodies are proteins that have the ability to specifically bind a particular antigen. Any antibody that can be expressed in a host cell can be used in accordance with the present invention. In one embodiment, the antibody to be expressed is a monoclonal antibody. In some embodiments, the antibody is an IgA, IgD, IgE, IgG, or IgM. In some embodiments, the antibody is an IgG1, IgG2, IgG3, or IgG4. In some embodiments, the antibody is a full antibody. In one embodiment, the antibody is a human IgG1 antibody.

Particular antibodies can be made, for example, by preparing and expressing synthetic genes that encode the recited amino acid sequences or by mutating human germline genes to provide a gene that encodes the recited amino acid sequences. Moreover, these antibodies can be produced, e.g., using one or more of the following methods. In some embodiments, the antibody is a chimeric antibody, humanized antibody or human antibody.

Numerous methods are available for obtaining antibodies, particularly human antibodies. One exemplary method includes screening protein expression libraries, e.g., phage or ribosome display libraries. Phage display is described, for example, U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809, each of which is incorporated herein by reference. The display of Fab's on phage is described, e.g., in U.S. Pat. Nos. 5,658,727; 5,667,988; and 5,885,793, each of which is incorporated herein by reference.

In addition to the use of display libraries, other methods can be used to obtain an antibody. For example, a protein or a peptide thereof can be used as an antigen in a non-human animal, e.g., a rodent, e.g., a mouse, hamster, or rat.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity can be produced and selected. See, e.g., XENOMOUSE™, Green et al. (1994) Nature Genetics 7:13-21, U.S. 2003-0070185, WO 96/34096, and WO 96/33735.

In another embodiment, an antibody is obtained from the non-human animal, and then modified, e.g., humanized or deimmunized. Winter describes an exemplary CDR-grafting method that can be used to prepare humanized antibodies described herein (U.S. Pat. No. 5,225,539, which is incorporated herein by reference). All or some of the CDRs of a particular human antibody can be replaced with at least a portion of a non-human antibody. In one embodiment, it is only necessary to replace the CDRs required for binding or binding determinants of such CDRs to arrive at a useful humanized antibody that binds to an antigen.

Humanized antibodies can be generated by replacing sequences of the Fv variable region that are not directly involved in antigen binding with equivalent sequences from human Fv variable regions. General methods for generating humanized antibodies are provided by Morrison, S. L. (1985) Science 229:1202-1207, by Oi et al. (1986) BioTechniques 4:214, and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, can be obtained from a hybridoma producing an antibody against a predetermined target, as described above, from germline immunoglobulin genes, or from synthetic constructs. The recombinant DNA encoding the humanized antibody can then be cloned into an appropriate expression vector. In one embodiment, the expression vector comprises a polynucleotide encoding a glutamine synthetase polypeptide. (See, e.g., Porter et al., Biotechnol Prog 26(5):1446-54 (2010).)

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237. Antibodies can have mutations in the CH2 region of the heavy chain that reduce or alter effector function, e.g., Fc receptor binding and complement activation. For example, antibodies can have mutations such as those described in U.S. Pat. Nos. 5,624,821 and 5,648,260. Antibodies can also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

In other embodiments, the antibody can be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies can be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem. 22:259-306. Removal of any carbohydrate moieties present on the antibodies can be accomplished chemically or enzymatically as described in the art (Hakimuddin et al. (1987) Arch. Biochem. Biophys. 259:52; Edge et al. (1981) Anal. Biochem. 118:131; and Thotakura et al. (1987) Meth. Enzymol. 138:350). See, e.g., U.S. Pat. No. 5,869,046 for a modification that increases in vivo half-life by providing a salvage receptor binding epitope.

The antibodies can be in the form of full length antibodies, or in the form of fragments of antibodies, e.g., Fab, F(ab′)₂, Fd, dAb, and scFv fragments. Additional forms include a protein that includes a single variable domain, e.g., a camel or camelized domain. See, e.g., U.S. 2005-0079574 and Davies et al. (1996) Protein Eng. 9(6):531-7.

In one embodiment, the antibody is an antigen-binding fragment of a full length antibody, e.g., a Fab, F(ab′)2, Fv or a single chain Fv fragment. Typically, the antibody is a full length antibody. The antibody can be a monoclonal antibody or a mono-specific antibody.

In another embodiment, the antibody can be a human, humanized, CDR-grafted, chimeric, mutated, affinity matured, deimmunized, synthetic or otherwise in vitro-generated antibody, and combinations thereof.

The heavy and light chains of the antibody can be substantially full-length. The protein can include at least one, and preferably two, complete heavy chains, and at least one, and preferably two, complete light chains) or can include an antigen-binding fragment (e.g., a Fab, F(ab′)2, Fv or a single chain Fv fragment). In yet other embodiments, the antibody has a heavy chain constant region chosen from, e.g., IgG1, IgG2, IgG3, IgG4, IgM, IgA1, IgA2, IgD, and IgE; particularly, chosen from, e.g., IgG1, IgG2, IgG3, and IgG4, more particularly, IgG1 (e.g., human IgG1). Typically, the heavy chain constant region is human or a modified form of a human constant region. In another embodiment, the antibody has a light chain constant region chosen from, e.g., kappa or lambda, particularly, kappa (e.g., human kappa).

Receptors

Another class of polypeptides that have been shown to be effective as pharmaceutical and/or commercial agents includes receptors. Receptors are typically trans-membrane glycoproteins that function by recognizing an extra-cellular signaling ligand. Receptors typically have a protein kinase domain in addition to the ligand recognizing domain, which initiates a signaling pathway by phosphorylating target intracellular molecules upon binding the ligand, leading to developmental or metabolic changes within the cell. In one embodiment, the receptors of interest are modified so as to remove the transmembrane and/or intracellular domain(s), in place of which there can optionally be attached an Ig-domain. In one embodiment, receptors to be produced in accordance with the present invention are receptor tyrosine kinases (RTKs). The RTK family includes receptors that are crucial for a variety of functions numerous in numerous cell types (see, e.g., Yarden and Ulrich, Ann. Rev. Biochem. 57:433-478 (1988); Ullrich and Schlessinger, Cell 61:243-254 (1990), incorporated herein by reference). Non-limiting examples of RTKs include members of the fibroblast growth factor (FGF) receptor family, members of the epithelial growth factor (EGF) family, platelet derived growth factor (PDGF) receptor, tyrosine kinase with immunoglobulin and EGF homology domains-1 (TIE-1) and TIE-2 receptors (Sato et al., Nature 376:70-74 (1995), incorporated herein by reference) and c-Met receptor, some of which have been suggested to promote angiogenesis, directly or indirectly (Mustonen and Alitalo, J. Cell Biol. 129:895-898 (1995)). Other non-limiting examples of RTKs include fetal liver kinase 1 (Terman et al., Oncogene 6:1677-83 (1991)) or vascular endothelial cell growth factor receptor 2 (VEGFR-2)), fins-like tyrosine kinase-1 (Flt-1) (DeVries et al., Science 255:989-991 (1992); Shibuya et al., Oncogene 5:519-524 (1990)), sometimes referred to as vascular endothelial cell growth factor receptor 1 (VEGFR-1), neuropilin-1, endoglin, endosialin, and Ax1. Those of ordinary skill in the art will be aware of other receptors that can be expressed in accordance with the present invention.

Growth Factors and Other Signaling Molecules

Another class of polypeptides that have been shown to be effective as pharmaceutical and/or commercial agents includes growth factors and other signaling molecules. Growth factors are typically glycoproteins that are secreted by cells and bind to and activate receptors on other cells, initiating a metabolic or developmental change in the receptor cell.

Non-limiting examples of mammalian growth factors and other signaling molecules include cytokines; epidermal growth factor (EGF), platelet-derived growth factor (PDGF); fibroblast growth factors (FGFs) such as aFGF and bFGF; transforming growth factors (TGFs) such as TGF-alpha and TGF-beta, including TGF-beta1, TGF-beta2, TGF-beta3, TGF-beta4, or TGF-beta5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (TLs), e.g., IL-1 to IL-10; tumor necrosis factor (TNF) alpha and beta; insulin A-chain; insulin B-chain; proinsulin; follicle stimulating hormone; calcitonin; luteinizing hormone; glucagon; anti-clotting factors such as Protein C; atrial natriuretic factor; lung surfactant; a plasminogen activator, such as urokinase or human urine or tissue-type plasminogen activator (t-PA); bombesin; thrombin, hemopoietic growth factor; enkephalinase; RANTES (regulated on activation normally T-cell expressed and secreted); human macrophage inflammatory protein (MIP-1-alpha); mullerian-inhibiting substance; relaxin A-chain; relaxin B-chain; prorelaxin; mouse gonadotropin-associated peptide; neurotrophic factors such as bone-derived neurotrophic factor (BDNF), neurotrophin-3, -4, -5, or -6 (NT-3, NT-4, NT-5, or NT-6), or a nerve growth factor such as NGF-beta. One of ordinary skill in the art will be aware of other growth factors or signaling molecules that can be expressed in accordance with the present invention.

Clotting Factors

In some embodiments, the protein of interest comprises a clotting factor. Clotting factor, as used herein, means any molecule, or analog thereof, which prevents or decreases the duration of a bleeding episode in a subject with a hemostatic disorder. For example, a clotting factor for the invention can be a full-length clotting factor, a mature clotting factor, or a chimeric clotting factor. In other words, it means any molecule having clotting activity. Clotting activity, as used herein, means the ability to participate in a cascade of biochemical reactions that culminates in the formation of a fibrin clot and/or reduces the severity, duration or frequency of hemorrhage or bleeding episode. Examples of clotting factors can be found in U.S. Pat. No. 7,404,956, which is herein incorporated by reference.

In one embodiment, the clotting factor is selected from Factor VII (FVII), FVIIa, Factor VIII (FVIII), Factor IX (FIX), FIXa (FIX), a Von Willebrand Factor (VWF) polypeptide, or any functional fragments thereof. In some embodiments, the chimeric clotting factor further comprises a heterologous moiety. In some embodiments, the heterologous moiety extends an in vivo half-life of the clotting factor. In some embodiments, the heterologous moiety is selected from the group consisting of albumin, albumin binding polypeptide, an FcRn binding partner, Fc, PAS, the β subunit of the C-terminal peptide (CTP) of human chorionic gonadotrophin, polyethylene glycol (PEG), hydroxyethyl starch (HES), albumin-binding small molecules, or combinations thereof.

In some embodiments, the FVIII is full-length FVIII or B-domain deleted FVIII. In some embodiments, the FVIII is single chain FVIII or dual chain FVIII.

In some embodiments, the recombinant polypeptide is a monomer-dimer hybrid. A monomer-dimer hybrid is a chimeric protein having a dimeric aspect and a monomeric aspect, wherein the dimeric aspect relates to the fact that it is comprised of two polypeptide chains each comprised of a portion of an immunoglobulin constant region, and wherein the monomeric aspect relates to the fact that only one of the two chains is comprised of a therapeutic biologically active molecule. Monomer-dimer hybrids are described in detail is U.S. Pat. No. 7,404,956, which is incorporated herein by reference in its entirety.

“Factor VII” or “FVII” refers to a coagulation factor protein synthesized in the liver and secreted into the blood as a single chain zymogen with a molecular weight of approximately 50 kDa. The FVII zymogen is converted into an activated form (FVIIa) by proteolytic cleavage. FVII is disclosed in U.S. Publ. No. 2011/0046061 and Int'l Publ. No. PCT/US2013/44842, each of which is incorporated herein by reference in its entirety. In some embodiments, the Factor VII polypeptide comprises inactivated Factor VII, active Factor VII (FVIIa), or activatable Factor VII.

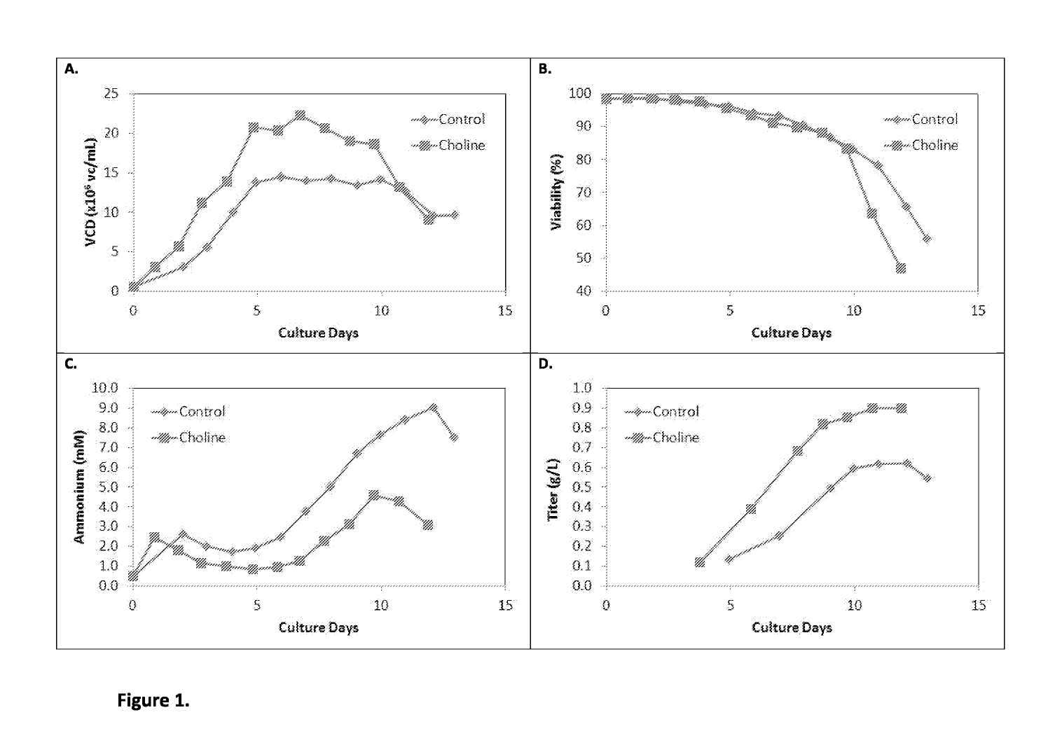

“Factor VIII” or “FVIII” refers to a blood coagulation factor protein and species and sequence variants thereof that includes, but is not limited to, the 2351 amino acid single-chain precursor protein (with a 19-amino acid hydrophobic signal peptide), the mature 2332 amino acid factor VIII protein of approximately 270-330 kDa with the domain structure A1-A2-B-A3-C1-C2, as well as the circulating heterodimer of two chains that form as a result of proteolytic cleavage after R1648 of a heavy chain form composed of A1-A2-B (in the range of 90-220 kD) of amino acids 1-1648 (numbered relative to the mature FVIII form) and a light chain A3-C1-C2 of 80 kDa of amino acids 1649-2232, each of which is depicted schematically in FIG. 1. “Factor VIII” or “FVIII” also can be sequence variants that retain at least a portion of the biological activity of the native circulating protein, including truncated sequences, a sequence that includes heterologous amino acids, or a single chain FVIII (scFVIII) in which the heavy and light chains are covalently connected by a linker. As used herein, “FVIII” shall be any functional form of factor VIII molecule with the typical characteristics of blood coagulation factor VIII capable of in vivo or in vitro correction of human factor VIII deficiencies (e.g., hemophilia A). FVIII or sequence variants have been isolated, characterized, and cloned, as described in U.S. Pat. or Publ. Nos. 4,757,006; 7,138,505; 5,004,804; 5,198,349, 5,250,421; 5,919,766; 2010/0081615; 2013/0017997 and 2013/0108629 each of which is incorporated herein by reference in its entirety. In some embodiments, the Factor VII polypeptide comprises full-length Factor VIII, mature Factor VIII, Factor VIII containing a partial or full deletion in B domain, or Factor VIII containing an insertion in one or more FVIII domains.

“B domain” of Factor VIII, as used herein, is the same as the B domain known in the art that is defined by internal amino acid sequence identity and sites of proteolytic cleavage by thrombin, e.g., residues Ser741-Arg1648 of full length human factor VIII. The other human factor VIII domains are defined by the following amino acid residues: A1, residues Ala1-Arg372; A2, residues Ser373-Arg740; A3, residues Ser1690-Ile2032; C1, residues Arg2033-Asn2172; C2, residues Ser2173-Tyr2332. The A3-C1-C2 sequence includes residues Ser1690-Tyr2332. The remaining sequence, residues Glu1649-Arg1689, is usually referred to as the factor VIII light chain activation peptide. The locations of the boundaries for all of the domains, including the B domains, for porcine, mouse and canine factor VIII are also known in the art. Preferably, the B domain of Factor VIII is deleted (“B domain deleted factor VIII” or “BDD FVIII”). An example of a BDD FVIII is REFACTO (recombinant BDD FVIII). The B domain of FVIII is discussed in U.S. Publ. No. 2013/0108629, which is incorporated herein by reference in its entirety.

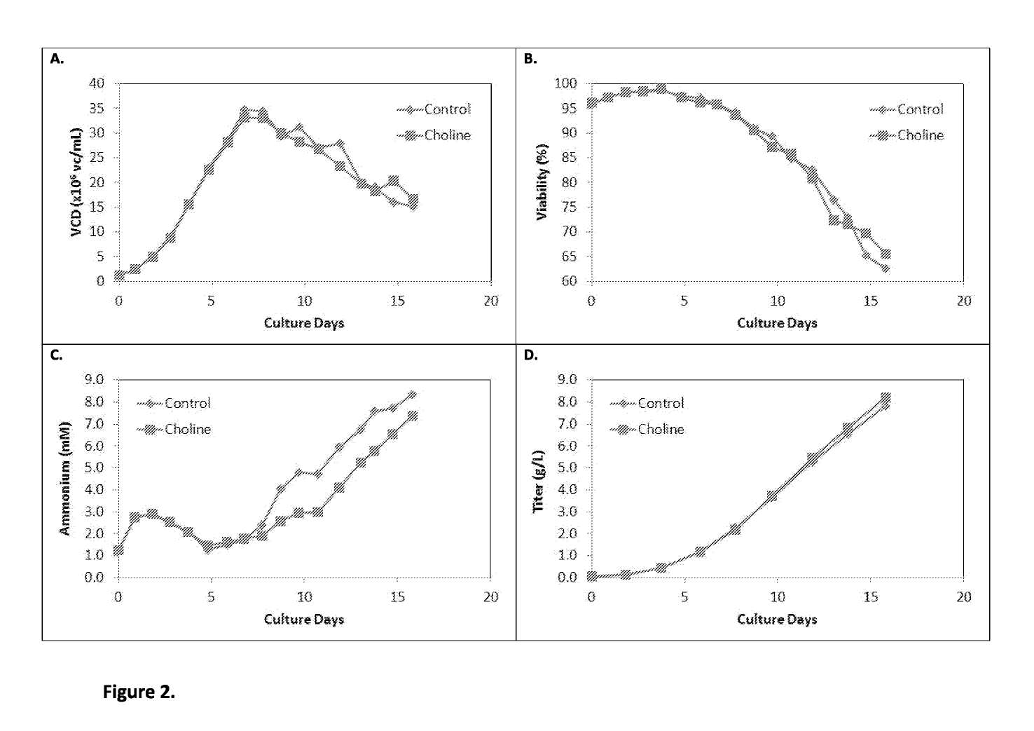

A “B domain deleted factor VIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563, each of which is incorporated herein by reference in its entirety. In some embodiments, a B domain deleted factor VIII sequence of the present invention comprises any one of the deletions disclosed at col. 4, line 4 to col. 5, line 28 and examples 1-5 of U.S. Pat. No. 6,316,226 (also in U.S. Pat. No. 6,346,513). In some embodiments, a B domain deleted factor VIII of the present invention has a deletion disclosed at col. 2, lines 26-51 and examples 5-8 of U.S. Pat. No. 5,789,203 (also U.S. Pat. No. 6,060,447, U.S. Pat. No. 5,595,886, and U.S. Pat. No. 6,228,620). In some embodiments, a B domain deleted factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563. In some embodiments, a B domain deleted factor VIII has a deletion of most of the B domain, but still contains amino-terminal sequences of the B domain that are essential for in vivo proteolytic processing of the primary translation product into two polypeptide chain, as disclosed in WO 91/09122, which is incorporated herein by reference in its entirety. In some embodiments, a B domain deleted factor VIII is constructed with a deletion of amino acids 747-1638, i.e., virtually a complete deletion of the B domain. Hoeben R. C., et al, J. Biol. Chem. 265 (13): 7318-7323 (1990), incorporated herein by reference in its entirety. A. B domain deleted factor VIII may also contain a deletion of amino acids 771-1666 or amino acids 868-1562 of factor VIII. Meulien P., et al. Protein Eng. 2(4): 301-6 (1988), incorporated herein by reference in its entirety. Additional B domain deletions that are part of the invention include, e.g.: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 through 1648 (Pasek (PCT application No. 88/00831)), 816 through 1598 or 741 through 1689 (Lagner (Behring Inst. Mitt. (1988) No 82:16-25, EP 295597)), each of which is incorporated herein by reference in its entirety. Each of the foregoing deletions may be made in any Factor VIII sequence. B domain deletions of FVIII are disclosed in U.S. Publ. No. 2013/0108629, which is incorporated herein by reference in its entirety.

“Factor IX” and “FIX,” as used herein, means functional Factor IX polypeptide in its normal role in coagulation, unless otherwise specified. Thus, the term Factor IX includes variant polypeptides that are functional and the polynucleotides that encode such functional variant polypeptides. Preferred Factor IX polypeptides are the human, bovine, porcine, canine, feline, and murine Factor IX polypeptides. The full length polypeptide and polynucleotide sequences of Factor IX are known, as are many functional variants, e.g., fragments, mutants and modified versions. Factor IX polypeptides include full-length Factor IX, full-length Factor IX minus Met at the N-terminus, full-length Factor IX minus the signal sequence, mature Factor IX (minus the signal sequence and propeptide), and mature Factor IX with an additional Met at the N-terminus. Factor IX is preferably made by recombinant means (“recombinant Factor IX” or “rFIX”), i.e., it is not naturally occurring or derived from plasma. FIX is disclosed in U.S. Publ. Nos. 2011/0046060 and 2013/0202595, each of which is incorporated herein by reference in its entirety.

VWF (also known as F8VWF) is a large multimeric glycoprotein present in blood plasma and produced constitutively in endothelium (in the Weibel-Palade bodies), megakaryocytes (α-granules of platelets), and subendothelian connective tissue. The basic VWF monomer is a 2813 amino acid protein. Every monomer contains a number of specific domains with a specific function, the D′/D3 domain (which binds to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (βHCG)).

The term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. The term “VWF fragment” as used herein does not include full length-or mature VWF protein.

G-Protein Coupled Receptors

Another class of polypeptides that have been shown to be effective as pharmaceutical and/or commercial agents includes growth factors and other signaling molecules. G-protein coupled receptors (GPCRs) are proteins that have seven transmembrane domains. Upon binding of a ligand to a GPCR, a signal is transduced within the cell which results in a change in a biological or physiological property of the cell.

GPCRs, along with G-proteins and effectors (intracellular enzymes and channels which are modulated by G-proteins), are the components of a modular signaling system that connects the state of intracellular second messengers to extracellular inputs. These genes and gene-products are potential causative agents of disease.

The GPCR protein superfamily now contains over 250 types of paralogues, receptors that represent variants generated by gene duplications (or other processes), as opposed to orthologues, the same receptor from different species. The superfamily can be broken down into five families: Family I, receptors typified by rhodopsin and the beta2-adrenergic receptor and currently represented by over 200 unique members; Family II, the recently characterized parathyroid hormone/calcitonin/secretin receptor family; Family III, the metabotropic glutamate receptor family in mammals; Family IV, the cAMP receptor family, important in the chemotaxis and development of D. discoideum; and Family V, the fungal mating pheromone receptors such as STE2.

Cells

Any eukaryotic cell or cell type susceptible to cell culture can be utilized in accordance with the present invention. For example, plant cells, yeast cells, animal cells, insect cells, avian cells or mammalian cells can be utilized in accordance with the present invention. In one embodiment, the eukaryotic cells are capable of expressing a recombinant protein.

Non-limiting examples of mammalian cells that can be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells ±DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCLS 1); TRI cells (Mather et al., Annals N.Y. Acad. Sci., 383:44-68 (1982)); MRC 5 cells; FS4 cells; and a human hepatoma line (Hep G2). In one embodiment, the present invention is used in the culturing of and expression of polypeptides from CHO cell lines. In a specific embodiment, the CHO cell line is the DG44 CHO cell line. In a specific embodiment, the CHO cell line is the DUXB11 CHO cell line. In a specific embodiment, the CHO cell line comprises a vector comprising a polynucleotide encoding a glutamine synthetase polypeptide. In a further specific embodiment, the CHO cell line expresses an exogenous glutamine synthetase gene. (See, e.g., Porter et al., Biotechnol Prog 26(5):1446-54 (2010).)

Additionally, any number of commercially and non-commercially available hybridoma cell lines that express polypeptides or proteins can be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

The eukaryotic cells according to the present invention can be selected or engineered to produce high levels of protein or polypeptide. Often, cells are genetically engineered to produce high levels of protein, for example by introduction of a gene encoding the protein or polypeptide of interest and/or by introduction of control elements that regulate expression of the gene (whether endogenous or introduced) encoding the polypeptide of interest.

The eukaryotic cells can also be selected or engineered to survive in culture for extended periods of time. For example, the cells can be genetically engineered to express a polypeptide or polypeptides that confer extended survival on the cells. In one embodiment, the eukaryotic cells comprise a transgene encoding the Bc1-2 polypeptide or a variant thereof. See, e.g., U.S. Pat. No. 7,785,880. In a specific embodiment, the cells comprise a polynucleotide encoding the bc1-xL polypeptide. See, e.g., Chiang G G, Sisk W P. 2005. Biotechnology and Bioengineering 91 (7): 779-792.

The eukaryotic cells can also be selected or engineered to modify its posttranslational modification pathways. In one embodiment, the cells are selected or engineered to modify a protein glycolsylation pathway. In a specific embodiment, the cells are selected or engineered to express an aglycosylated protein, e.g., an aglycosylated recombinant antibody. In another specific embodiment, the cells are selected or engineered to express an afucosylated protein, e.g., an afucosylated recombinant antibody.

The eukaryotic cells can also be selected or engineered to allow culturing in serum free medium.

Media

The cell culture of the present invention is prepared in any medium suitable for the particular cell being cultured. In some embodiments, the medium contains e.g., inorganic salts, carbohydrates (e.g., sugars such as glucose, galactose, maltose or fructose), amino acids, vitamins (e.g., B group vitamins (e.g., B12), vitamin A vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. Commercially available media such as 5×-concentrated DMEM/F12 (Invitrogen), CD OptiCHO feed (Invitrogen), CD EfficientFeed (Invitrogen), Cell Boost (HyClone), BalanCD CHO Feed (Irvine Scientific), BD Recharge (Becton Dickinson), Cellvento Feed (EMD Millipore), Ex-cell CHOZN Feed (Sigma-Aldrich), CHO Feed Bioreactor Supplement (Sigma-Aldrich), SheffCHO (Kerry), Zap-CHO (Invitria), ActiCHO (PAA/GE Healthcare), Ham's F10 (Sigma), Minimal Essential Medium ([MEM], Sigma), RPMI-1640 (Sigma), and Dulbecco's Modified Eagle's Medium ([DMEM], Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range) lipids (such as linoleic or other fatty acids) and their suitable carriers, and glucose or an equivalent energy source. In some embodiments the nutrient media is serum-free media, a protein-free media, or a chemically defined media. Any other necessary supplements can also be included at appropriate concentrations that would be known to those skilled in the art.

In one embodiment, the mammalian host cell is a CHO cell and a suitable medium contains a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) with modified concentrations of some components such as amino acids, salts, sugar, and vitamins, recombinant human insulin, hydrolyzed peptone, such as Primatone HS or Primatone RL (Sheffield, England), or the equivalent; a cell protective agent, such as Pluronic F68 or the equivalent pluronic polyol; gentamycin; and trace elements.

The present invention provides a variety of media formulations that, when used in accordance with other culturing steps described herein, minimize, prevent or reverse metabolic imbalances in the culture that would lead to increased lactate and ammonium production.

A media formulation of the present invention that has been shown to have beneficial effects on metabolic balance, cell growth, and/or viability or on expression of polypeptide or protein comprise hypotaurine, Gamma-Aminobutyric Acid (GABA), and/or beta-alanine or the combination of choline with hypotaurine, GABA, and/or beta-alanine. One of ordinary skill in the art will understand that the media formulations of the present invention encompass both defined and non-defined media.

Cell Culture Processes

Various methods of preparing mammalian cells for production of proteins or polypeptides by batch and fed-batch culture are well known in the art. See Kshirsagar et al., Biotechnology Bioengineering 109:2523-2532 (2012). A nucleic acid sufficient to achieve expression (typically a vector containing the gene encoding the polypeptide or protein of interest and any operably linked genetic control elements) can be introduced into the host cell line by any number of well-known techniques. Typically, cells are screened to determine which of the host cells have actually taken up the vector and express the polypeptide or protein of interest. Traditional methods of detecting a particular polypeptide or protein of interest expressed by mammalian cells include but are not limited to immunohistochemistry, immunoprecipitation, flow cytometry, immunofluorescence microscopy, SD S-PAGE, Western blots, enzyme-linked immunosorbentassay (ELISA), high performance liquid chromatography (HPLC) techniques, biological activity assays and affinity chromatography. One of ordinary skill in the art will be aware of other appropriate techniques for detecting expressed polypeptides or proteins. If multiple host cells express the polypeptide or protein of interest, some or all of the listed techniques can be used to determine which of the cells expresses that polypeptide or protein at the highest levels.

Once a cell that expresses the polypeptide or protein of interest has been identified, the cell is propagated in culture by any of the variety of methods well-known to one of ordinary skill in the art. The cell expressing the polypeptide of interest is typically propagated by growing it at a temperature and in a medium that is conducive to the survival, growth and viability of the cell. The initial culture volume can be of any size, but is often smaller than the culture volume of the production bioreactor used in the final production of the polypeptide or protein of interest, and frequently cells are passaged several times in bioreactors of increasing volume prior to seeding the production bioreactor. The cell culture can be agitated or shaken to increase oxygenation of the medium and dispersion of nutrients to the cells. Alternatively or additionally, special sparging devices that are well known in the art can be used to increase and control oxygenation of the culture. In accordance with the present invention, one of ordinary skill in the art will understand that it can be beneficial to control or regulate certain internal conditions of the bioreactor, including but not limited to pH, temperature, oxygenation, etc.

The cell density useful in the methods of the present invention can be chosen by one of ordinary skill in the art. In accordance with the present invention, the cell density can be as low as a single cell per culture volume. In some embodiments of the present invention, starting cell densities (seed density) can range from about 2×10² viable cells per mL to about 2×10³, 2×10⁴, 2×10⁵, 2×10⁶, 5×10⁶, 10×10⁶, 20×10⁶, 30×10⁶, or 40×10⁶ viable cells per mL and higher.

In accordance with the present invention, a cell culture size can be any volume that is appropriate for production of polypeptides. In one embodiment, the volume of the cell culture is at least 500 liters. In other embodiments, the volume of the production cell culture is 10, 50, 100, 250, 1000, 2000, 2500, 5000, 8000, 10,000, 12,000 liters or more, or any volume in between. For example, a cell culture will be 10 to 5,000 liters, 10 to 10,000 liters, 10 to 15,000 liters, 50 to 5,000 liters, 50 to 10,000 liters, or 50 to 15,000 liters, 100 to 5,000 liters, 100 to 10,000 liters, 100 to 15,000 liters, 500 to 5,000 liters, 500 to 10,000 liters, 500 to 15,000 liters, 1,000 to 5,000 liters, 1,000 to 10,000 liters, or 1,000 to 15,000 liters. Or a cell culture will be between about 500 liters and about 30,000 liters, about 500 liters and about 20,000 liters, about 500 liters and about 10,000 liters, about 500 liters and about 5,000 liters, about 1,000 liters and about 30,000 liters, about 2,000 liters and about 30,000 liters, about 3,000 liters and about 30,000 liters, about 5,000 liters and about 30,000 liters, or about 10,000 liters and about 30,000 liters, or a cell culture will be at least about 500 liters, at least about 1,000 liters, at least about 2,000 liters, at least about 3,000 liters, at least about 5,000 liters, at least about 10,000 liters, at least about 15,000 liters, or at least about 20,000 liters.

One of ordinary skill in the art will be aware of and will be able to choose a suitable culture size for use in practicing the present invention. The production bioreactor for the culture can be constructed of any material that is conducive to cell growth and viability that does not interfere with expression or stability of the produced polypeptide or protein.

The temperature of the cell culture will be selected based primarily on the range of temperatures at which the cell culture remains viable. For example, during the initial growth phase, CHO cells grow well at 37° C. In general, most mammalian cells grow well within a range of about 25° C. to 42° C.

In one embodiment of the present invention, the temperature of the initial growth phase is maintained at a single, constant temperature. In another embodiment, the temperature of the initial growth phase is maintained within a range of temperatures. For example, the temperature can be steadily increased or decreased by discrete amounts at various times during the initial growth phase. One of ordinary skill in the art will be able to determine whether a single or multiple temperatures should be used, and whether the temperature should be adjusted steadily or by discrete amounts.

The cells can be grown during the initial growth phase for a greater or lesser amount of time, depending on the needs of the practitioner and the requirement of the cells themselves. In one embodiment, the cells are grown for a period of time sufficient to achieve a viable cell density that is a given percentage of the maximal viable cell density that the cells would eventually reach if allowed to grow undisturbed. For example, the cells can be grown for a period of time sufficient to achieve a desired viable cell density of 1, 5, 10, 15, 20, 25, 30, 35, 40, 45, 50, 55, 60, 65, 70, 75, 80, 85, 90, 95, or 99 percent of maximal viable cell density.

In another embodiment, the cells are allowed to grow for a defined period of time. For example, depending on the starting concentration of the cell culture, the temperature at which the cells are grown, and the intrinsic growth rate of the cells, the cells can be grown for 0, 1, 2, 3, 4, 5, 6, 7, 8, 9, 10, 11, 12, 13, 14, 15, 16, 17, 18, 19, 20, or more days. In some cases, the cells can be allowed to grow for a month or more. In one embodiment, the growth phase is between about 1 day and about 20 days, about 1 day and about 15 days, about 1 day and about 14 days, about 1 day and about 13 days, about 1 day and about 12 days, about 1 day and about 11 days, about 1 day and about 10 days, about 1 day and about 10 days, about 1 day and about 9 days, about 1 day and about 8 days, about 1 day and about 7 days, about 1 day and about 6 days, about 1 day and about 5 days, about 1 day and about 4 days, about 1 day and about 3 days, about 2 days and about 15 days, about 3 days and about 15 days, about 4 days and about 15 days, about 5 days and about 15 days, about 6 days and about 15 days, about 7 days and about 15 days, about 8 days and about 15 days, about 9 days and about 15 days, about 10 days and about 15 days, about 2 days and about 20 days, about 3 days and about 20 days, about 4 days and about 20 days, about 5 days and about 20 days, about 6 days and about 20 days, about 7 days and about 20 days, about 8 days and about 20 days, about 9 days and about 20 days, about 10 days and about 20 days, or about 15 days and about 20 days. In another embodiment, the growth phase is at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 15 days, or at least about 20 days. In a further embodiment, the growth phase is about 1 day, about 2 days, about 3 days, about 4 days, about 5 days, about 6 days, about 7 days, about 8 days, about 9 days, about 10 days, about 11 days, about 12 days, about 15 days, or about 20 days.

The cells would be grown for 0 days in the production bioreactor if their growth in a seed bioreactor, at the initial growth phase temperature, was sufficient that the viable cell density in the production bioreactor at the time of its inoculation is already at the desired percentage of the maximal viable cell density. The practitioner of the present invention will be able to choose the duration of the initial growth phase depending on polypeptide or protein production requirements and the needs of the cells themselves.

The cell culture can be agitated or shaken during the initial culture phase in order to increase oxygenation and dispersion of nutrients to the cells. In accordance with the present invention, one of ordinary skill in the art will understand that it can be beneficial to control or regulate certain internal conditions of the bioreactor during the initial growth phase, including but not limited to pH, temperature, oxygenation, etc. For example, pH can be controlled by supplying an appropriate amount of acid or base and oxygenation can be controlled with sparging devices that are well known in the art.

The temperature of the cell culture in the subsequence growth phase will be selected based primarily on the range of temperatures at which the cell culture remains viable and expresses recombinant polypeptides or proteins at commercially adequate levels. In general, most mammalian cells remain viable and express recombinant polypeptides or proteins at commercially adequate levels within a range of about 25° C. to 42° C. In one embodiment, mammalian cells remain viable and express recombinant polypeptides or proteins at commercially adequate levels within a range of about 25° C. to 35° C. Those of ordinary skill in the art will be able to select appropriate temperature or temperatures in which to grow cells, depending on the needs of the cells and the production requirements of the practitioner.

In accordance with the present invention, the cells can be maintained in the subsequent production phase until a desired cell density or production titer is reached. In one embodiment, the cells are maintained in the subsequent production phase until the titer to the recombinant polypeptide or protein reaches a maximum. In other embodiments, the culture can be harvested prior to this point, depending on the production requirement of the practitioner or the needs of the cells themselves. For example, the cells can be maintained for a period of time sufficient to achieve a viable cell density of 1, 5, 10, 15, 20, 25, 30, 35, 40, 45, 50, 55, 60, 65, 70, 75, 80, 85, 90, 95, or 99 percent of maximal viable cell density. In some cases, it is desirably to allow the viable cell density to reach a maximum, and then allow the viable cell density to decline to some level before harvesting the culture. In an extreme example, it can be desirable to allow the viable cell density to approach or reach zero before harvesting the culture.

In another embodiment of the present invention, the cells are allowed to grow for a defined period of time during the subsequent production phase. For example, depending on the concentration of the cell culture at the start of the subsequent growth phase, the temperature at which the cells are grown, and the intrinsic growth rate of the cells, the cells can be grown for 1, 2, 3, 4, 5, 6, 7, 8, 9, 10, 11, 12, 13, 14, 15, 16, 17, 18, 19, 20, or more days. In some cases, the cells can be allowed to grow for a month or more. The practitioner of the present invention will be able to choose the duration of the subsequent production phase depending on polypeptide or protein production requirements and the needs of the cells themselves.

In certain cases, it can be beneficial or necessary to supplement the cell culture during the growth and/or subsequent production phase with nutrients or other medium components that have been depleted or metabolized by the cells. For example, it might be advantageous to supplement the cell culture with nutrients or other medium components observed to have been depleted. Alternatively or additionally, it can be beneficial or necessary to supplement the cell culture prior to the subsequent production phase. As non-limiting examples, it can be beneficial or necessary to supplement the cell culture with hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, or glucose or other energy source. In some embodiments, the cell culture is supplemented with hypotaurine, Gamma-Aminobutyric Acid (GABA), and/or beta-alanine or the combination of choline with hypotaurine, GABA, and/or beta-alanine.

These supplementary components, including the amino acids, can all be added to the cell culture at one time, or they can be provided to the cell culture in a series of additions. In one embodiment of the present invention, the supplementary components are provided to the cell culture at multiple times in proportional amounts. In another embodiment, it can be desirable to provide only certain of the supplementary components initially, and provide the remaining components at a later time. In yet another embodiment of the present invention, the cell culture is fed continually with these supplementary components.

In accordance with the present invention, the total volume added to the cell culture should optimally be kept to a minimal amount. For example, the total volume of the medium or solution containing the supplementary components added to the cell culture can be 1, 2, 3, 4, 5, 6, 7, 8, 9, 10, 15, 20, 25, 30, 35, 40, 45 or 50% of the volume of the cell culture prior to providing the supplementary components.

The cell culture can be agitated or shaken during the subsequent production phase in order to increase oxygenation and dispersion of nutrients to the cells. In accordance with the present invention, one of ordinary skill in the art will understand that it can be beneficial to control or regulate certain internal conditions of the bioreactor during the subsequent growth phase, including but not limited to pH, temperature, oxygenation, etc. For example, pH can be controlled by supplying an appropriate amount of acid or base and oxygenation can be controlled with sparging devices that are well known in the art.

In certain embodiments of the present invention, the practitioner can find it beneficial or necessary to periodically monitor particular conditions of the growing cell culture. Monitoring cell culture conditions allows the practitioner to determine whether the cell culture is producing recombinant polypeptide or protein at suboptimal levels or whether the culture is about to enter into a suboptimal production phase.

In order to monitor certain cell culture conditions, it will be necessary to remove small aliquots of the culture for analysis. One of ordinary skill in the art will understand that such removal can potentially introduce contamination into the cell culture, and will take appropriate care to minimize the risk of such contamination.

As non-limiting example, it can be beneficial or necessary to monitor temperature, pH, cell density, cell viability, integrated viable cell density, lactate levels, ammonium levels, osmolarity, or titer of the expressed polypeptide or protein. Numerous techniques are well known in the art that will allow one of ordinary skill in the art to measure these conditions. For example, cell density can be measured using a hemacytometer, a Coulter counter, or Cell density examination (CEDEX). Viable cell density can be determined by staining a culture sample with Trypan blue. Since only dead cells take up the Trypan blue, viable cell density can be determined by counting the total number of cells, dividing the number of cells that take up the dye by the total number of cells, and taking the reciprocal. HPLC can be used to determine the levels of lactate, ammonium or the expressed polypeptide or protein. Alternatively, the level of the expressed polypeptide or protein can be determined by standard molecular biology techniques such as coomassie staining of SDS-PAGE gels, Western blotting, Bradford assays, Lowry assays, Biuret assays, and UV absorbance. It can also be beneficial or necessary to monitor the post-translational modifications of the expressed polypeptide or protein, including phosphorylation and glycosylation.

The practitioner can also monitor the metabolic status of the cell culture, for example, by monitoring the glucose, lactate, ammonium, and amino acid concentrations in the cell culture, as well as by monitoring the oxygen production or carbon dioxide production of the cell culture. For example, cell culture conditions can be analyzed by using NOVA Bioprofile 100 or 400 (NOVA Biomedical, WA). Additionally, the practitioner can monitor the metabolic state of the cell culture by monitoring the activity of mitochondria. In one embodiment, mitochondrial activity can be monitored by monitoring the mitochondrial membrane potential using Rhodamine 123. Johnson L V, Walsh M L, Chen L B. 1980. Proceedings of the National Academy of Sciences 77(2): 990-994.

Isolation of Expressed Polypeptide

In general, it will typically be desirable to isolate and/or purify proteins or polypeptides expressed according to the present invention. In one embodiment, the expressed polypeptide or protein is secreted into the medium and thus cells and other solids can be removed, as by centrifugation or filtering for example, as a first step in the purification process.

Alternatively, the expressed polypeptide can be bound to the surface of the host cell. In this embodiment, the media is removed and the host cells expressing the polypeptide or protein are lysed as a first step in the purification process. Lysis of mammalian host cells can be achieved by any number of means well known to those of ordinary skill in the art, including physical disruption by glass beads and exposure to high pH conditions.

The polypeptide can be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, the protein can be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin can be added at any or all stages in order to reduce or eliminate degradation of the polypeptide or protein during the purification process. Protease inhibitors are particularly desired when cells must be lysed in order to isolate and purify the expressed polypeptide or protein. One of ordinary skill in the art will appreciate that the exact purification technique will vary depending on the character of the polypeptide or protein to be purified, the character of the cells from which the polypeptide or protein is expressed, and the composition of the medium in which the cells were grown.

Pharmaceutical Compositions

A polypeptide can be formulated as a pharmaceutical composition for administration to a subject, e.g., to treat or prevent a disorder or disease. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (See e.g., Berge, S. M., et al. (1977) J. Pharm. Sci. 66:1-19).

Pharmaceutical formulation is a well-established art, and is further described, e.g., in Gennaro (ed.), Remington. The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions can be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. Typically compositions for the agents described herein are in the form of injectable or infusible solutions.

In one embodiment, the antibody is formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the polypeptide can be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

The foregoing description is to be understood as being representative only and is not intended to be limiting. Alternative methods and materials for implementing the invention and also additional applications will be apparent to one of skill in the art, and are intended to be included within the accompanying claims.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C.A.K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical—Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D., Immunology 6th ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein, are incorporated herein by reference in their entireties.

### Examples

As discussed above, one of the primary barriers to achieving mammalian fed-batch cell cultures that are both long and productive is the accumulation of growth- and protein production-inhibitory metabolic waste byproducts, such as ammonium and lactate. Studies were carried out to determine the effects of hypotaurine, Gamma-Aminobutyric Acid (GABA), and choline on the waste byproduct accumulation of two cell lines. It was found that addition of hypotaurine and/or choline can reduce ammonium accumulation. It was found that addition of GABA can reduce lactate accumulation. Addition of hypotaurine and choline in tandem can reduce the accumulation of both ammonium and lactate. The main benefit of cultures exhibiting reduced ammonium and lactate profiles is that these conditions yield longer-lasting and more productive (higher titer) fed-batch cell culture processes.

### Example 1—Effect of Choline on Cell Line A

The impact of different choline levels in the culture on cell density, viability, ammonium concentration and titer was evaluated in Cell Line A (FIG. 1). Cell Line A expressed the polypeptide Neublastin. The control feed medium contained 3 mM choline chloride. The choline feed medium contained 9 mM choline chloride. Feed medium was added daily from Day 2 to Day 12 to the cell culture. As can be seen in FIG. 1, the choline feed medium condition exhibited higher growth and viability (A, B), lower ammonium accumulation (C), and higher titer (D) as compared to the control condition. Initial cell density was one million cells (1e6). The effects of choline were evident starting on Day 3.

TABLE 1
Effective choline concentration at Days 3 and 13
in Cell Line A cultures treated with control
or choline feed medium.

| | Day 13 | Day 3 (Effective) |
|---|---|---|
| | Choline Conc. | Choline Conc. |
| Cell Line A | (mM) | (mM) |
| Control | 1.45 mM | 0.67 mM |
| Choline | 3.32 mM | 0.90 mM |

### Example 2—Effect of Choline on Cell Line B

The impact of different choline levels in the culture on cell density, viability, ammonium concentration and titer was evaluated in Cell Line B (FIG. 2). Cell Line B expressed the polypeptide Lingo. The control feed medium contained 3 mM choline chloride. The choline feed medium contained 18 mM choline chloride. Feed medium was added daily from Day 1 to Day 15 to the cell culture. As can be seen in FIG. 2, the choline feed medium condition exhibited higher growth and viability (A, B), lower ammonium accumulation (C), and slightly higher titer (D) as compared to the control condition. Initial cell density was one million cells (1e6). The effects of choline were evident starting on Day 9.

TABLE 2
Effective choline concentration at Days 9 and 16
in Cell Line B cultures treated with control
or choline feed medium.

| | Day 16 | Day 9 (Effective) |
|---|---|---|
| | Choline Conc. | Choline Conc. |
| Cell Line B | (mM) | (mM) |
| Control | 1.1 mM | 0.78 mM |
| Choline | 6.6 mM | 4.65 mM |

### Example 3—Effect of Hypotaurine on Cell Line B

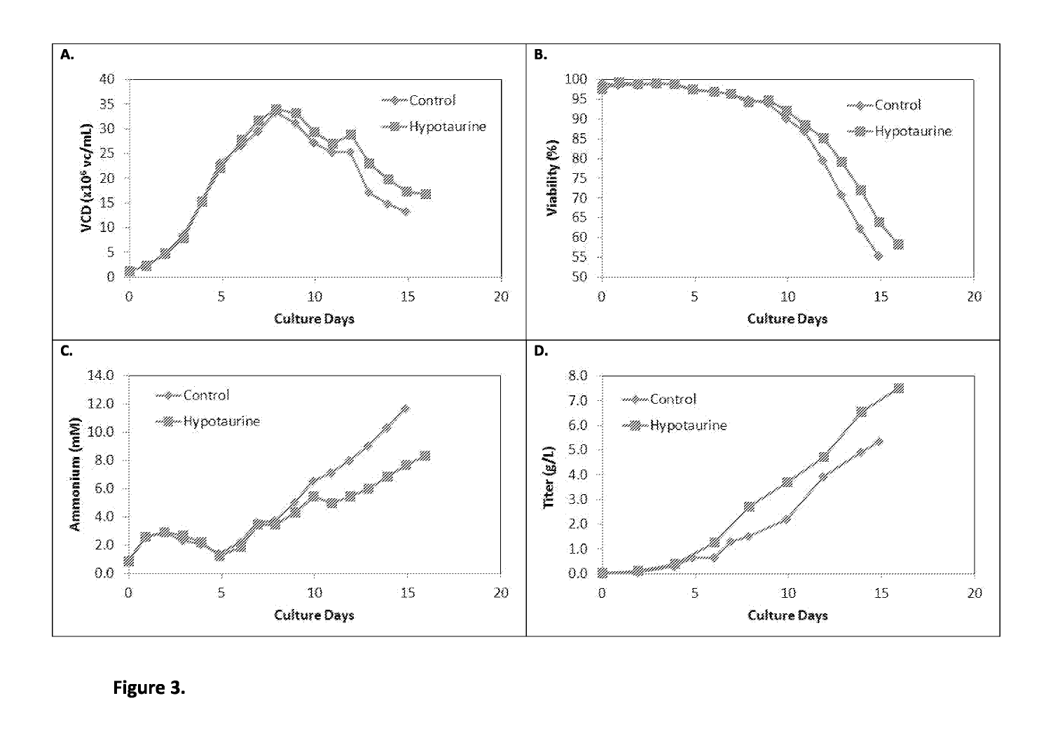

The impact of different hypotaurine levels in the culture on cell density, viability, ammonium concentration and titer was evaluated in Cell Line B (FIG. 3). The control feed medium contained 0 mM hypotaurine. The hypotaurine feed medium contained 8 mM hypotaurine. Feed medium was added daily from Day 1 to Day 15 to the cell culture. The concentration of hypotaurine in the bioreactor on Day 16 was approximately 2.7 mM. The hypotaurine feed medium condition exhibited higher growth and viability (A, B), lower ammonium accumulation (C), and higher titer (D) than the control feed medium condition. Initial cell density was one million cells (1e6). The effects of hypotaurine were evident starting on Day 10.

TABLE 3
Effective hypotaurine concentration at Days 10 and 16
in Cell Line B cultures treated with control
or hypotaurine feed medium.

| | Day 16 | Day 10 (Effective) |
|---|---|---|
| | Hypotaurine Conc. | Hypotaurine Conc. |
| Cell Line B | (mM) | (mM) |
| Control | 0 mM | 0 mM |
| Hypotaurine | 3.18 mM | 2.5 mM |

### Example 4—Combined Effect of Hypotaurine and Choline on Cell Line B, Feed Regime 1

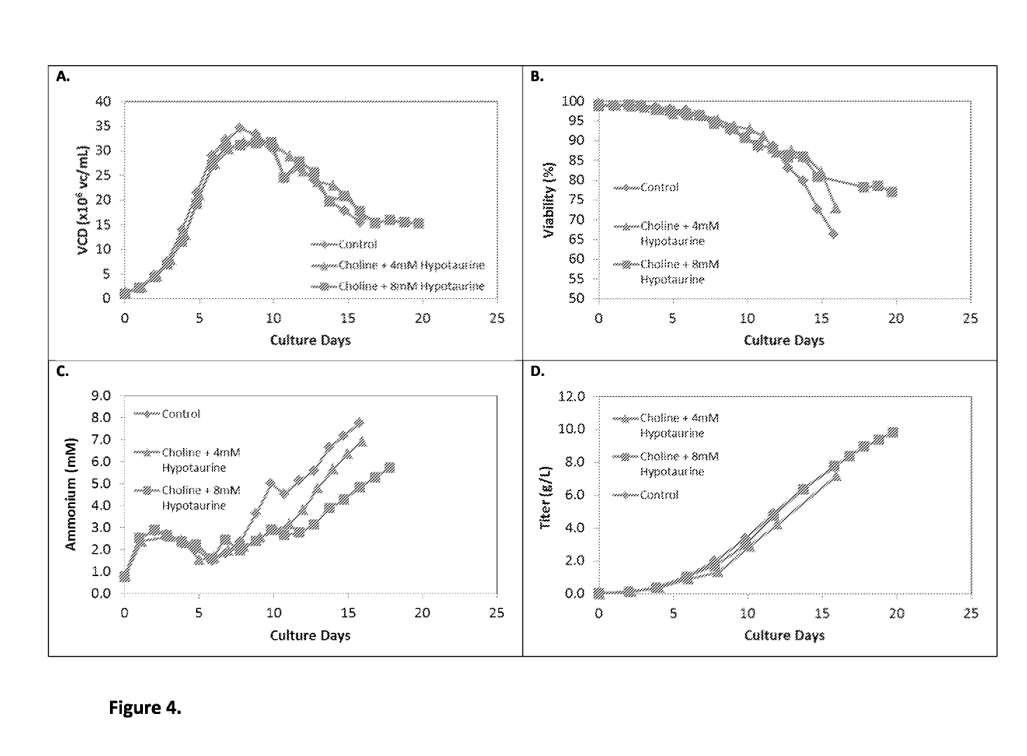

The impact of different hypotaurine and choline levels in the culture on cell density, viability, ammonium concentration and titer was evaluated in Cell Line B (FIG. 4). The control feed medium contained 3 mM choline and 0 mM hypotaurine. The combined hypotaurine and choline feed media contained 18 mM choline chloride and 4 mM hypotaurine in one instance (“4 mM hypotaurine”) and 18 mM choline chloride and 8 mM hypotaurine in a second instance (“8 mM hypotaurine”). Initial cell density was one million cells (1e6). Feed medium was added daily from Day 1 to Day 15 for the control condition, daily from Day 1 to Day 15 for the 4 mM hypotaurine condition and daily from Day 1 to Day 19 for the 8 mM hypotaurine condition.

The 8 mM hypotaurine feed medium condition exhibited higher growth and viability (A, B) and lower ammonium accumulation (C) than the control condition. The 4 mM hypotaurine condition had little effect on growth, viability, and titer (A, B, D) when compared to the control condition. The higher viability and lower ammonium concentration associated with the 8 mM hypotaurine in the feed medium allowed the culture duration to be prolonged to Day 20, which resulted in the realization of higher final titers (D).

TABLE 4
Effective choline and hypotaurine concentrations in Cell Line B cultures
treated with control or choline/hypotaurine feed medium.

| | Final | Final | Effective | Effective |
|---|---|---|---|---|
| | Choline | Hypotaurine | Choline | Hypotaurine |
| | Conc. | Conc. | Conc. | Conc. |
| Cell Line B | (mM) | (mM) | (mM) | (mM) |
| Control | 1.1 mM | 0 mM | 0.47 mM | 0 mM |
| | (Day 16) | | (Day 7) | |
| Choline + 4 mM | 6.7 mM | 1.5 mM | 5.7 mM | 1.28 mM |
| Hypotaurine | (Day 16) | (Day 16) | (Day 12) | (Day 12) |
| Choline + 8 mM | 7.2 mM | 3.18 mM | 2.8 mM | 1.24 mM |
| Hypotaurine | (Day 20) | (Day 20) | (Day 7) | (Day 7) |

### Example 5—Combined Effect of Hypotaurine and Choline on Cell Line B, Feed Regime 2

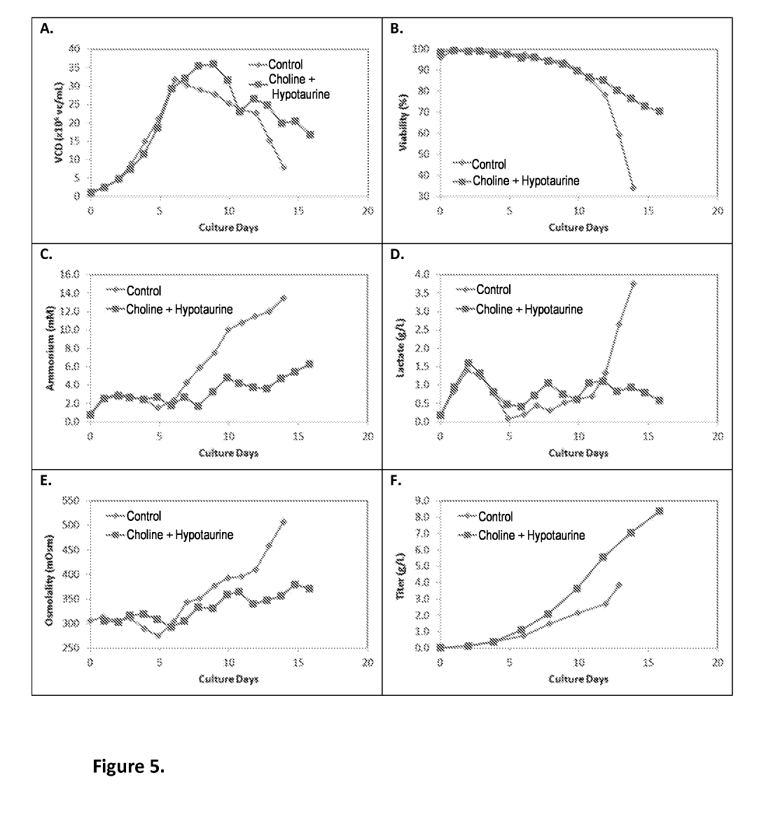

The impact of different hypotaurine and choline levels in the culture on cell density, viability, ammonium concentration and titer was evaluated in Cell Line B (FIG. 5). Initial cell density was one million cells (1e6). The control feed medium contained 3 mM choline chloride and 0 mM hypotaurine. The combined choline and hypotaurine feed medium contained 18 mM choline chloride and 8 mM hypotaurine. Feed medium was added daily from Day 1 to Day 15. The combined hypotaurine and choline condition exhibited higher growth and viability (A, B) and lower ammonium, lactate, and osmolality accumulation (C, D, E), while the control condition viability crashed early due to toxic accumulation of waste and subsequently osmolality. The overall healthier culture and increased feeding regime allowed titers to be increased to >8 g/L on Day 16 (F).

TABLE 5
Effective choline and hypotaurine concentrations in
Cell Line B cultures treated with control or choline/hypotaurine
feed medium at Days 13 and 16.

| | Final | Final | Effective | Effective |
|---|---|---|---|---|
| | Choline | Hypotaurine | Choline | Hypotaurine |
| | Conc. | Conc. | Conc. | Conc. |
| Cell Line B | (mM) | (mM) | (mM) | (mM) |
| Control | 1.3 mM | 0 mM | 1.1 mM | 0 mM |
| | (Day 16) | | (Day 13) | |
| Choline + 8 mM | 7.8 mM | 3.45 mM | 6.8 mM | 3.04 mM |
| Hypotaurine | (Day 16) | (Day 16) | (Day 13) | (Day 13) |

### Example 6—Effect of Alternative Antioxidants on Cell Line B Waste Accumulation and Cell Culture Performance

Studies were performed on the precursor and by-product of hypotaurine as well as other antioxidants to determine if an alternative chemical could provide the same benefit as hypotaurine.

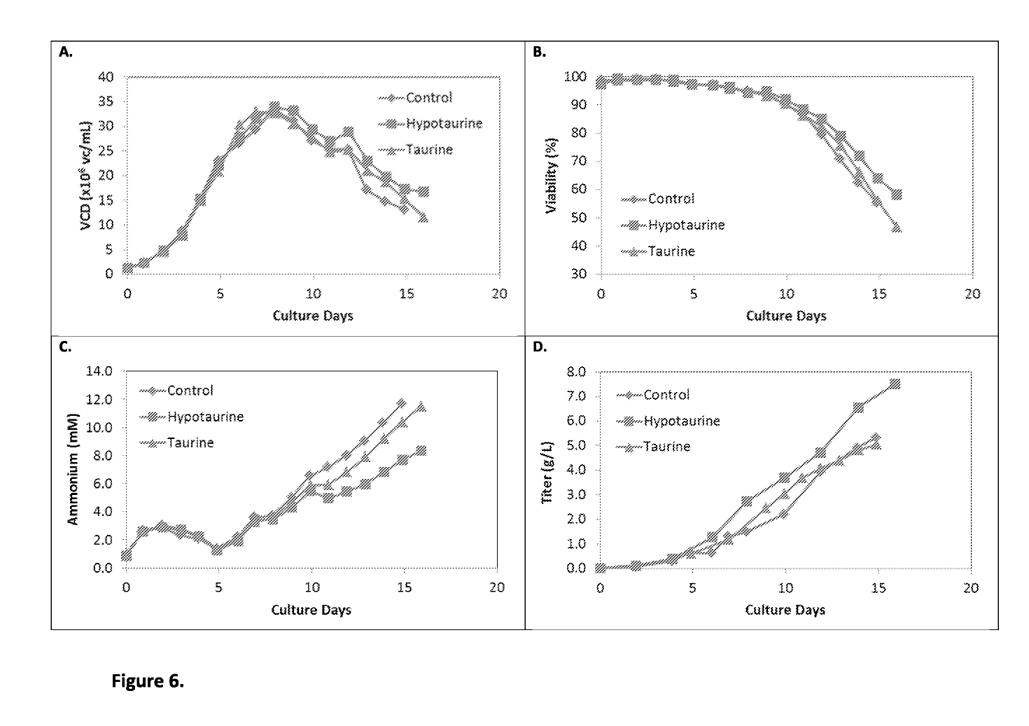

Taurine is the by-product of hypotaurine metabolism. Therefore, cell culture performance when 8 mM taurine was added to the feed medium was compared to cell culture performance when 8 mM of hypotaurine was added to the feed medium. Initial cell density was one million cells (1e6). As can be seen in FIG. 6, despite being the downstream product of hypotaurine, taurine was not able to serve as a replacement for hypotaurine.

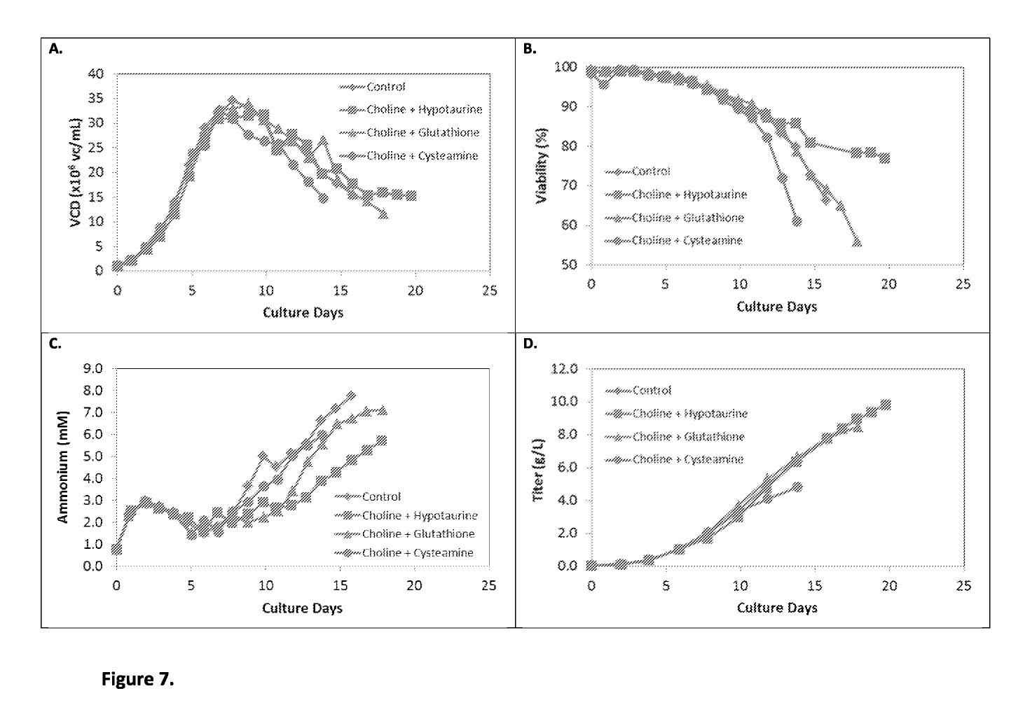

Cysteamine is a precursor of hypotaurine. Therefore, cell culture performance when 3 mM cysteamine and 18 mM choline chloride were added to the feed medium was compared to cell culture performance when 8 mM hypotaurine and 18 mM choline chloride were added to the feed medium. Initial cell density was one million cells (1e6). The effects of glutathione, another type of antioxidant, were also explored. The feed medium for the glutathione condition contained 1.3 mM glutathione and 18 mM choline chloride. Neither cysteamine nor glutathione were able to serve as a replacement for hypotaurine (FIG. 7).

### Example 7—Combined Effect of Hypotaurine and Choline on Cell Line B High Seed Fed-Batch Process

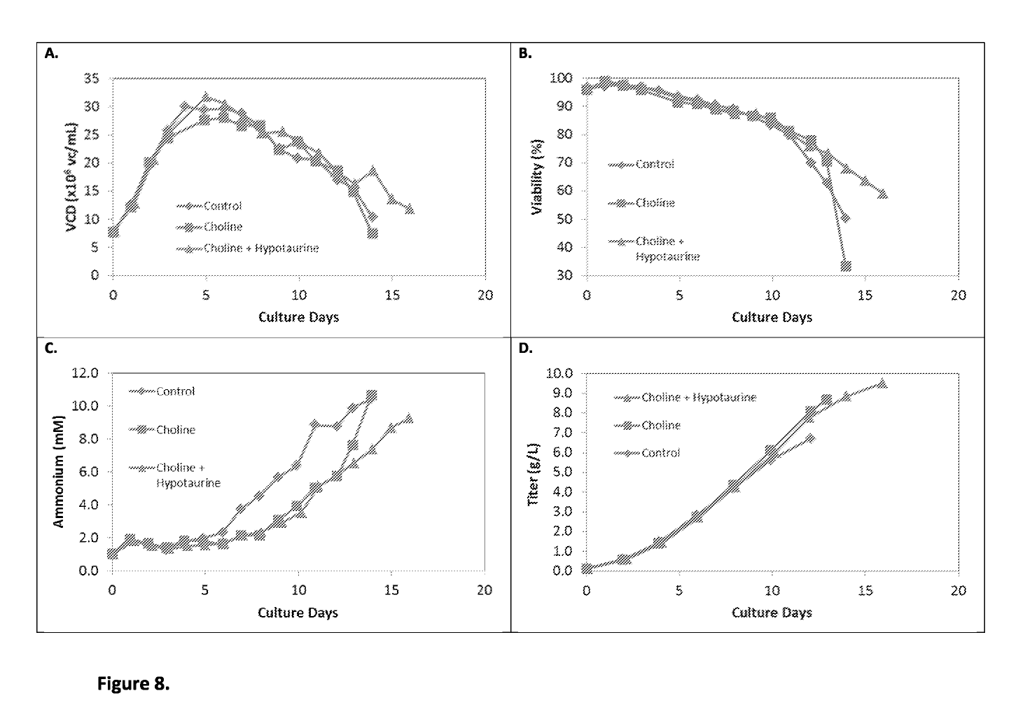

The impact of different hypotaurine and choline levels in the culture on cell density, viability, ammonium concentration and titer was evaluated in Cell Line B in a high seed fed-batch processes (FIG. 8).

Hypotaurine and choline were tested on the Cell Line B high seed fed-batch process, where the initial seeding density was 8×10⁶ viable cells/mL (vc/mL) compared to 1×10⁶ vc/mL. The control feed medium contained 3 mM choline chloride and 0 mM hypotaurine. The choline-only feed medium contained 18 mM choline chloride and 0 mM hypotaurine. The combined choline and hypotaurine feed medium contained 18 mM choline chloride and 8 mM hypotaurine.

A maximal mitigation of waste accumulation was obtained by using a combination of choline and hypotaurine. The combination of choline and hypotaurine prevented the culture viability crash that occurred after day 12 for the control and choline-only conditions (FIG. 8B). The addition of hypotaurine and choline was associated with higher growth and viability and lower ammonium accumulation (FIGS. 8A, 8B, and 8C). The higher viability and lower ammonium allowed the culture duration to be prolonged to Day 16 and higher final titers to be realized (8D).

TABLE 6
Effective choline and hypotaurine concentrations in
Cell Line B cultures in a high seed fed-batch process treated
with control, choline, or choline/hypotaurine feed medium

| | Final | Final | Effective | Effective |
|---|---|---|---|---|
| | Choline | Hypotaurine | Choline | Hypotaurine |
| | Conc. | Conc. | Conc. | Conc. |
| Cell Line B | (mM) | (mM) | (mM) | (mM) |
| Control | 1.1 mM | 0 mM | 0.67 mM | 0 mM |
| | (Day 12) | | (Day 6) | |
| | | | 1.2 mM | |
| | | | (Day 13) | |
| Choline | 7.1 mM | 0 mM | 4.0 mM | 0 mM |
| | (Day 12) | | (Day 6) | |
| Choline + | 8.0 mM | 3.56 mM | 4.1 mM | 1.8 mM |
| Hypotaurine | (Day 16) | (Day 16) | (Day 6) | (Day 6) |
| | | | 7.5 mM | 3.36 mM |
| | | | (Day 13) | (Day 13) |

### Example 8—Effect of Hypotaurine and Choline on Cell Line B High Seed Fed-Batch Process-Alternate Final Concentrations

Hypotaurine and choline were tested on the Cell Line B regular seed (seed density of 1e6) and high seed fed-batch process (seed density of 10e6). The feed medium contained 18 mM choline and 8 mM hypotaurine for both processes and both processes were fed daily. Duration was 14 days. The feeding strategy was capacitance-based. The high seed process fed about 80% of the initial working volume of the bioreactor. As before, the effective concentration of choline when used alone was 4.65 mM and the effective concentration of hypotaurine when used alone was 2.5 mM. In addition, the other parameters were as shown below. The effective concentration based on the ammonia difference was measured on day 6.

TABLE 7A

| | Final |
|---|---|
| | Concentration |
| Condition | (mM) |
| Cell Line B (1e6) Hypotaurine | 3.5 mM |
| Cell Line B (1e6) Choline | 7.8 mM |
| Cell Line B High Seed (10e6) | 3.75 mM |
| Hypotaurine | |
| Cell Line B High Seed (10e6) Choline | 8.44 mM |

TABLE 7B

| | Final | Final | Effective | Effective |
|---|---|---|---|---|
| | Choline | Hypotaurine | Choline | Hypotaurine |
| | Conc. | Conc. | Conc. | Conc. |
| Cell Line B | (mM) | (mM) | (mM) | (mM) |
| Control | 1.3 mM | 0 mM | 0.35 mM | 0 mM |
| | (Day 16) | | (Day 6) | |
| | | | 1.1 mM | |
| | | | (Day 13) | |
| Choline + 8 mM | 7.8 mM | 3.45 mM | 2.13 mM | 0.95 mM |
| Hypotaurine | (Day 16) | (Day 16) | (Day 6) | (Day 6) |
| | | | 6.8 mM | 3.04 mM |
| | | | (Day 13) | (Day 13) |

The effective choline and hypotaurine concentrations were the same as for 8e6 seed process.

### Example 9—Effect of GABA on Growth, Viability and Lactate Concentration

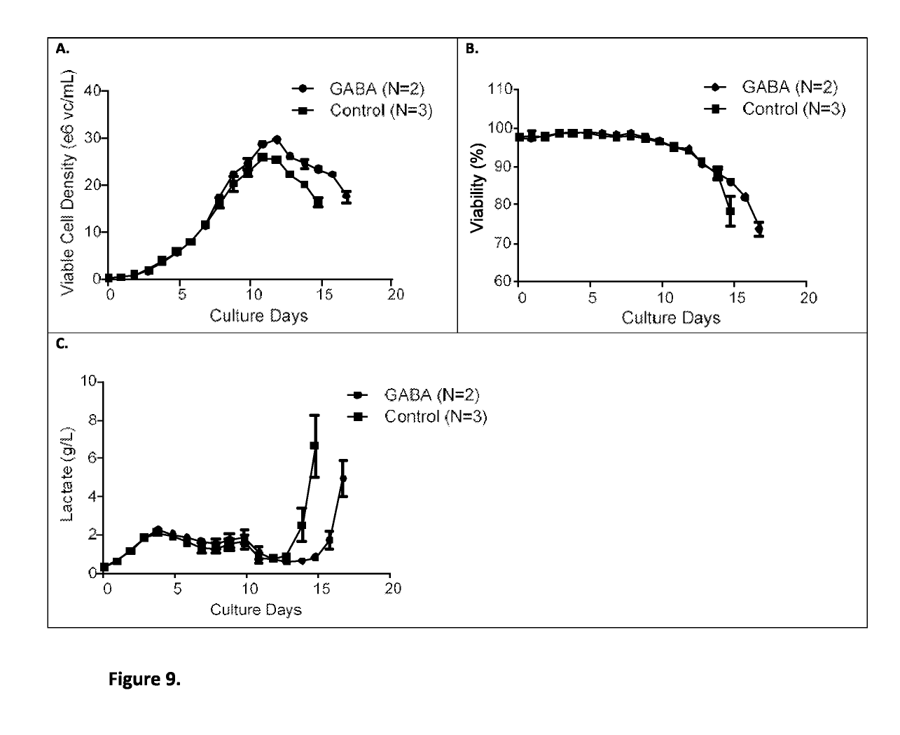

The impact of gamma-aminobutyric acid (GABA) levels in the culture on cell density (growth), viability, and lactate concentration was evaluated in CHO cells in a fed-batch process (FIG. 9).

Cryopreserved CHO cells were thawed and maintained in 500 mL, 1000 mL or 3000 mL shake flasks with 100 mL, 200 mL or 1000 mL working volumes using cell culture medium CM3 and were passaged every 2 to 4 days. For maintenance cultures, the incubator was controlled at 36° C. and 5% CO₂. A 5 liter (L) glass Applikon vessels using Finesse TruBio DV controllers (Finesse Solutions, San Jose, Calif.) were used for the fed-batch process. Bioreactors were seeded with an initial working volume of 2.5 L at a constant seed. For the control process, feed medium, CF2b, was given on day 3, day 5 and daily thereafter (starting on day 6) until the day before harvest and culture termination. The control process lasted 15 days with harvest and culture termination on day 15. CF2b is designated as a complete feed, comprised of all necessary nutritional elements including glucose, amino acids, vitamins, and metals. For GABA culture, feed medium was supplemented with 8 mM of GABA. The GABA culture lasted 17 days with harvest and culture termination on day 17. The complete feed amount was calculated as a predetermined fixed percentage based on culture volume. Glucose is maintained by the feeding strategy above 2 g/L in general by the bolus feeds and is not limiting. Culture temperature was set at 35° C. and medium pH was controlled at 7.1±0.2 by the addition of either 1 M sodium carbonate or CO₂ gas. Dissolved oxygen (DO) was maintained at 30% with air sparge, enriched with oxygen as necessary, through a drilled hole sparger. Agitation was maintained between 200 and 400 rpm throughout the culture to limit total gas flow. An air overlay was maintained between 0.005 and 0.02 gas volume flow per unit of liquid volume per minute (vvm). Both control and GABA cultures had a seeding density of 3e5 cells/ml.

Supplementation with GABA was associated with higher growth and viability and lower lactate accumulation (FIGS. 9A, 9B, and 9C). The higher viability and lower lactate accumulation allowed the culture duration to be prolonged. GABA supplementation delayed the onset of a high lactate phenotype by about 2 days compared to control.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All documents, articles, publications, patents, and patent applications mentioned in this specification are herein incorporated by reference to the same extent as if each individual publication or patent application was specifically and individually indicated to be incorporated by reference.

What is claimed is:

1. A method of producing a polypeptide of interest in a large-scale cell culture, comprising culturing mammalian cells expressing the polypeptide of interest in a cell culture medium under conditions that support expression of the polypeptide of interest, wherein said cell culture medium comprises hypotaurine, Gamma-Aminobutyric Acid (GABA), and/or beta-alanine.

2. A method of producing a polypeptide of interest in a large-scale cell culture, comprising supplementing the culture with a feed medium comprising a sufficient amount of hypotaurine, Gamma-Aminobutyric Acid (GABA), or beta-alanine to achieve a hypotaurine, GABA, or beta-alanine concentration, respectively, in the culture between about 0.1 mM and 500 mM, wherein the culture comprises cells expressing the polypeptide and a medium, and the cells are maintained under conditions that allow for expression of the polypeptide.

3. A method of producing a polypeptide of interest in a large-scale cell culture, comprising:
a) providing cells capable of expressing the polypeptide and a hypotaurine-, Gamma-Aminobutyric Acid (GABA)-, or beta-alanine-containing cell culture medium;
b) supplementing the culture with a feed medium comprising a sufficient amount of hypotaurine, GABA, or beta-alanine to achieve a hypotaurine, GABA, or beta-alanine concentration, respectively, of between about 0.1 mM to 500 mM; and
c) culturing the cells of b) to allow for expression of the polypeptide.

4. The method of claim 1, wherein the culture medium comprises between about 0.1 mM and about 500 mM hypotaurine, GABA, or beta-alanine.

5. The method of any one of claims 1-4, wherein the medium further comprises choline.

6. The method of any one of claims 1-5, further comprising supplementing the culture with a feed medium comprising a sufficient amount of hypotaurine, GABA, or beta-alanine to maintain the hypotaurine, GABA, or beta-alanine concentration, respectively, in the culture to between about 0.1 mM and about 500 mM.

7. The method of any one of claims 1-6, wherein the feed medium comprises hypotaurine, GABA, or beta-alanine in an amount sufficient to achieve a hypotaurine, GABA, or beta-alanine concentration, respectively, in the culture of between about 0.1 mM and about 500 mM, between about 0.1 mM and about 400 mM, between about 0.1 mM and about 300 mM, between about 0.1 mM and about 200 mM, between about 0.1 mM and about 100 mM, between about 0.1 mM and about 50 mM, between about 0.1 mM and about 25 mM, between about 10 mM and about 500 mM, between about 20 mM and about 500 mM, between about 50 mM and about 500 mM, between about 100 mM and about 500 mM, between about 200 mM and about 500 mM, between about 10 mM and about 100 mM, between about 50 mM and about 200 mM, between about 100 mM and about 400 mM, between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

8. The method of any one of claims 1-7, wherein the feed medium comprises choline in an amount sufficient to achieve a choline concentration in the culture of between about 0.1 mM and about 10 mM, about 0.1 mM and about 9 mM, about 0.1 mM and about 8 mM, about 0.1 mM and about 7 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.1 mM and about 0.5 mM, about 0.5 mM and about 10 mM, about 1 mM and about 10 mM, about 2 mM and about 10 mM, about 3 mM and about 10 mM, about 4 mM and about 10 mM, about 5 mM and about 10 mM, about 6 mM and about 10 mM, about 7 mM and about 10 mM, about 8 mM and about 10 mM, about 9 mM and about 10 mM, about 2 mM and about 5 mM, about 4 mM and about 7 mM, about 6 mM and about 9 mM, about 3 mM and about 6 mM, about 4 mM and about 7 mM, or about 5 mM and about 8 mM.

9. The method of any one of claims 1-8, wherein the cells are maintained in a cell culture medium containing hypotaurine, GABA, or beta-alanine at a concentration of about 0.1 mM to 500 mM for between about 1 day and about 20 days.

10. The method of claim 9, wherein the cells are maintained in a cell culture medium containing hypotaurine, GABA, or beta-alanine at a concentration of about 0.1 mM to 500 mM for between about 1 day and about 20 days, about 1 day and about 15 days, about 1 day and about 10 days, about 1 day and about 9 days, about 1 day and about 8 days, or about 1 day and about 7 days.

11. The method of claim 10, wherein the cell culture medium at the hypotaurine, GABA, or beta-alanine concentration is maintained for at least about 1 day, at least about 2 days, at least about 3 days, at least about 4 days, at least about 5 days, at least about 6 days, at least about 7 days, at least about 8 days, at least about 9 days, at least about 10 days, at least about 15 days, or at least about 20 days.

12. The method of any one of claims 1-11, wherein the culture is supplemented with the feed medium between about 1 and about 20 times.

13. The method of claim 12, wherein the culture is supplemented with the feed medium about 1 and about 20 times, between about 1 and about 15 times, or between about 1 and about 10 times.

14. The method of claim 13, wherein the culture is supplemented with the feed medium at least once, at least twice, at least three times, at least four times, at least five times, at least six times, at least seven times, at least eight times, at least nine times, at least ten times, at least 11 times, at least 12 times, at least 13 times, at least 14 times, at least 15 times, or at least 20 times.

15. The method of any one of claims 1-14, wherein the lactate production of the cells is lower than the lactate production of cells maintained in a culture medium that is substantially free from choline and hypotaurine, GABA, or beta-alanine, respectively.

16. The method of claim 15, wherein the lactate production of the cells is between about 5% and about 95% lower than the lactate production of cells maintained in a culture medium that is substantially free from choline and hypotaurine, GABA, or beta-alanine, respectively.

17. The method of claim 16, wherein the lactate production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than the lactate production of cells maintained in a culture medium that is substantially free of choline and hypotaurine, GABA, or beta-alanine, respectively.

18. The method of any one of claims 1-17, wherein the lactate concentration of the culture is between about 0.1 g/L and about 10 g/L.

19. The method of claim 18, wherein the lactate concentration of the culture is between about 0.1 g/L and about 5 g/L, between about 0.1 g/L and about 4 g/L, or between about 0.1 g/L and about 3 g/L.

20. The method of claim 19, wherein the lactate concentration of the culture is less than about 6 g/L, about 5 g/L, about 4 g/L, about 3 g/L, about 2 g/L, or about 1 g/L.

21. The method of any one of claims 1-20, wherein the ammonium production of the cells is lower than the ammonium production of cells maintained in a culture medium that is substantially free of choline and hypotaurine, GABA, or beta-alanine, respectively.

22. The method of claim 21, wherein the ammonium production of the cells is between about 5% and about 90% lower than the ammonium production of cells maintained in a culture medium that is substantially free from choline and hypotaurine, GABA, or beta-alanine, respectively.

23. The method of claim 22, wherein the ammonium production of the cells is between about 5% and about 80%, between about 5% and about 70%, between about 5% and about 50%, between about 5% and about 40%, between about 5% and about 30%, between about 5% and about 20%, between about 10% and about 90%, between about 20% and about 90%, between about 30% and about 90%, or between about 50% and about 90% lower than ammonium production of cells maintained in a culture medium that is substantially free from choline and hypotaurine, GABA, or beta-alanine, respectively.

24. The method of any one of claims 1-23, wherein the ammonium concentration of the culture is between about 0.1 mM and about 20 mM.

25. The method of claim 24, wherein the ammonium concentration of the culture is between about 0.1 mM and about 15 mM, about 0.1 mM and about 14 mM, about 0.1 mM and about 13 mM, about 0.1 mM and about 12 mM, about 0.1 mM and about 11 mM, about 0.1 mM and about 10 mM, about 0.1 mM and about 6 mM, about 0.1 mM and about 5 mM, about 0.1 mM and about 4 mM, about 0.1 mM and about 3 mM, about 0.1 mM and about 2 mM, about 0.1 mM and about 1 mM, about 0.5 mM and about 15 mM, about 0.5 mM and about 14 mM, about 0.5 mM and about 13 mM, about 0.5 mM and about 12 mM, about 0.5 mM and about 11 mM, about 0.5 mM and about 10 mM, about 0.5 mM and about 9 mM, about 0.5 mM and about 8 mM, about 0.5 mM and about 7 mM, about 0.5 mM and about 6 mM, about 0.5 mM and about 5 mM, about 0.5 mM and about 4 mM, about 0.5 mM and about 3 mM, about 0.5 mM and about 2 mM, about 0.5 mM and about 1 mM, about 1 mM and about 15 mM, about 1 mM and about 14 mM, about 1 mM and about 13 mM, about 1 mM and about 12 mM, about 1 mM and about 11 mM, about 1 mM and about 10 mM, about 1 mM and about 9 mM, about 1 mM and about 8 mM, about 1 mM and about 7 mM, about 1 mM and about 6 mM, about 1 mM and about 5 mM, about 1 mM and about 4 mM, about 1 mM and about 3 mM, or about 1 mM and about 2 mM.

26. The method of claim 25, wherein the ammonium concentration of the culture is less than about 20 mM, about 19 mM, about 18 mM, about 17 mM, about 16 mM, about 15 mM, about 14 mM, about 13 mM, about 12, mM, about 11 mM, about 10 mM, about 9 mM, about 8 mM, about 7 mM, about 6 mM, about 5 mM, about 4 mM, about 3 mM, about 2 mM, about 1 mM, or about 0.5 mM.

27. The method of any one of claims 1-26, wherein the cell specific lactate production rate to the cell specific glucose uptake rate ratio (LPR/GUR ratio) of the cells is between about −0.5 and about 0.5.

28. The method of claim 27, wherein the LPR/GUR ratio of the cells is between about −0.4 and about 0.5, about −0.3 and about 0.5, about −0.2 and about 0.5, about −0.1 and about 0.5, about −0.5 and about 0.4, about −0.5 and about 0.3, about −0.5 and about 0.2, about −0.5 and about 0.1, about −0.4 and about 0.4, about −0.3 and about 0.3, about −0.2 and about 0.2, about −0.1 and about 0.1, about −0.1 and about 0.5, about −0.2 and about 0.1, or about −0.3 and about 0.1.

29. The method of any one of claims 1-28, wherein the cells are selected from the group consisting of CHO cells, HEK cells, NS0 cells, PER.C6 cells, HeLa cells, and MDCK cells.

30. The method of claim 29, wherein the cells are CHO cells.

31. The method of claim 29, wherein the cells are HEK cells.

32. The method of any one of claims 1-28, wherein the cells are hybridoma cells.

33. The method of any one of claims 1-32, wherein the cells have been adapted to grow in serum free medium, animal protein free medium or chemically defined medium.

34. The method of any one of claims 1-33, wherein the cells have been genetically modified to alter their innate glycosylation pathways.

35. The method of any one of claims 1-34, wherein the cells have been genetically modified to increase their life-span in culture.

36. The method of any one of claims 1-35, wherein the total amount of polypeptide produced by the cells is higher than the total amount of polypeptide produced by cells maintained in a culture medium that is substantially free from choline and hypotaurine, GABA, or beta-alanine, respectively.

37. The method of claim 36, wherein the total amount of polypeptide produced by the cell is between about 5% and about 500% higher than the total amount of polypeptide produced by the cells maintained in a culture medium that is substantially free from choline and hypotaurine, GABA, or beta-alanine, respectively.

38. The method of claim 37, wherein the total amount of polypeptide produced by the cell is between about 5% and about 300% higher than the total amount of polypeptide produced by the cells maintained in a culture medium that is substantially free from choline and hypotaurine, GABA, or beta-alanine, respectively.

39. The method of any one of claims 1-38, wherein the specific productivity of the cells is higher than the specific productivity of cells maintained in a culture medium that is substantially free of choline and hypotaurine, GABA, or beta-alanine, respectively.

40. The method of claim 39, wherein the specific productivity of the cells is between about 5% and about 500% higher than the specific productivity of cells maintained in a culture medium that is substantially free from choline and hypotaurine, GABA, or beta-alanine, respectively.

41. The method of claim 40, wherein the specific productivity of the cells is between about 5% and about 300% higher than the specific productivity of cells maintained in the culture medium that is substantially free from choline and hypotaurine, GABA, or beta-alanine, respectively.

42. The method of any one of claims 1-41, wherein the culture is a perfusion culture.

43. The method of any one of claims 1-41, wherein the culture is a fed batch culture.

44. The method of any one of claims 1-41, wherein the culture is conducted in a shake flask.

45. The method of any one of claims 1-41, wherein the culture is conducted in a stirred-tank bioreactor.

46. The method of any one of claims 1-45, wherein the cell culture has a volume between about 500 liters and about 30,000 liters.

47. The method of any one of claims 1-46, wherein the medium is a serum free medium, animal protein free medium, or a chemically defined medium.

48. The method of claim 47, wherein the medium is a chemically defined medium.

49. The method of any one of claims 1-48, wherein the hypotaurine, GABA, or beta-alanine is introduced into the culture medium as part of a feed medium.

50. The method of any one of claims 1-48, wherein hypotaurine, GABA, or beta-alanine is introduced into the culture medium as one or more boli from a distinct stock solution.

51. The method of any one of claims 1-50, wherein the choline is introduced into the culture medium as part of a feed medium.

52. The method of any one of claims 1-50, wherein the choline is introduced into the culture medium as one or more boli from a distinct stock solution.

53. The method of any one of claims 1-52, wherein the polypeptide of interest is selected from the group consisting of: an antibody, a Transforming Growth Factor (TGF) beta superfamily signaling molecule, an Fc fusion protein, interferon beta-1a, Lingo, and a clotting factor.

54. The method of any one of claims 1-53, wherein the polypeptide of interest is an antibody.

55. The method of claim 54, wherein the antibody is an IgA, IgD, IgE, IgG, or IgM.

56. The method of claim 54 or 55, wherein the antibody is an IgG1, IgG2, IgG3, or IgG4.

57. The method of any one of claims 54-56, wherein the antibody is a full antibody.

58. The method of any one of claims 54-57, wherein the antibody is a chimeric antibody, humanized antibody or human antibody.

59. The method of any one of claims 54-58, wherein the antibody is a human IgG1 antibody.

60. The method of any one of claims 54-59, wherein the antibody is an anti-α4-integrin antibody.

61. The method of claim 60, wherein the antibody is natalizumab.

62. The method of claim 54-59, wherein the antibody is an anti-TWEAK antibody.

63. The method of claims 54-59, wherein the antibody is an anti-LINGO antibody.

64. The method of claims 54-59, wherein the antibody is anti-amyloid beta antibody.

65. The method of claims 54-59, wherein the antibody is an anti-CD20 antibody.

66. The method of claim 65, wherein the antibody is rituximab.

67. The method of claim 65, wherein the antibody is obinutuzumab.

68. The method of claims 54-59, wherein the antibody is an anti-IL2 antibody.

69. The method of claim 68, wherein the antibody is daclizumab.

70. The method of claims 54-59, wherein the antibody is an anti-αvβ6 integrin antibody.

71. The method of claims 54-59, wherein the antibody is an anti-tau antibody.

72. The method of claim 53, wherein said TGF-beta superfamily signaling molecule is Neublastin.

73. The method of claim 53, wherein said clotting factor is a full-length clotting factor, a mature clotting factor, or a chimeric clotting factor.

74. The method of claim 53 or 73, wherein the clotting factor comprises a Factor VIII polypeptide, a Factor VII polypeptide, a Factor IX polypeptide, a Von Willebrand Factor polypeptide, or any functional fragments thereof.

75. The method of claim 74, wherein the clotting factor further comprises a heterologous moiety.

76. The method of claim 75, wherein the heterologous moiety extends an in vivo half-life of the clotting factor.

77. The method of claim 75, wherein the heterologous moiety is selected from the group consisting of albumin, albumin binding polypeptide, an FcRn binding partner, Fc, PAS, the β subunit of the C-terminal peptide (CTP) of human chorionic gonadotrophin, polyethylene glycol (PEG), hydroxyethyl starch (HES), albumin-binding small molecules, or combinations thereof.

78. The method of any one of claim 53 or 73-77, wherein the clotting factor is a monomer-dimer hybrid.

79. The method of any one of claim 53 or 74-77, wherein the Factor VII polypeptide comprises inactivated Factor VII, active Factor VII (FVIIa), or activatable Factor VII.

80. The method of any one of claim 53 or 74-77, wherein the Factor VIII polypeptide comprises full-length Factor VIII, mature Factor VIII, Factor VIII containing a partial or full deletion in B domain, or Factor VIII containing an insertion in one or more FVIII domains.

81. The method of claim 53, wherein the polypeptide of interest is interferon beta-1a.

82. The method of claim 53, wherein the polypeptide of interest is CD40L.

83. The method of any one of claims 1-4, wherein the culture medium comprises between about 0.1 mM and about 500 mM hypotaurine.

84. The method of any one of claims 1-4, wherein the culture medium comprises between about 0.1 mM and about 500 mM GABA.

85. The method of any one of claim 84, wherein the culture medium comprises between about 3 mM and about 20 mM GABA.

86. The method of any one of claims 1-4, wherein the culture medium comprises between about 0.1 mM and about 500 mM beta-alanine.

87. The method of any one of claims 1-4, wherein the culture medium comprises hypotaurine, GABA, and beta-alanine.

88. The method of claim 88, wherein the medium further comprises choline.
